SEQUENCE LISTING (2 sequences)

SEQ ID NO: 1 - protein, 130 aa, Homo sapiens
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 2 - DNA, 394 bp, Homo sapiens
ggggtgagcgaaactgcaccagcgagtcgtcggggtgagctggctgtgtgcgatgcagtcagtggctgggtgacagaccgccggaccgctgtggacttgcgtgggcgcgaggtggaggtgttgggcgaggtgcctgcagctggcggcagtcccctccgccagtacttctttgaaacccgctgcaaggctgataacgctgaggaaggtggcccgggggcaggtggagggggctgccggggagtggacaggaggcactgggtatctgagtgcaaggccaagcagtcctatgtgcgggcattgaccgctgatgcccagggccgtgtgggctggcgatggattcgaattgacactgcctgcgtctgcacactcctcagccggactggccgggcctgag

(19) United States
(12) Patent Application Publication
(10) Pub. No.: US 2009/0291897 A1
(43) Pub. Date: Nov. 26, 2009

(54) METHODS FOR TREATING UNWANTED WEIGHT LOSS OR EATING DISORDERS BY ADMINISTERING A TRKB AGONIST

(75) Inventors: Chia-Yang Lin, Palo Alto, CA (US); Arnon Rosenthal, Woodside, CA (US); Jennifer Renee Stratton, Belmont, CA (US)

Correspondence Address: PFIZER INC, 10555 SCIENCE CENTER DRIVE, SAN DIEGO, CA, 92121, US

(73) Assignee: RINAT NEUROSCIENCE CORPORATION

(21) Appl. No.: 12/508,379

(22) Filed: Jul. 23, 2009

Related U.S. Application Data
(63) Continuation of application No. 11/670,096, filed on Feb. 1, 2007.
(60) Provisional application No. 60/765,410, filed on Feb. 2, 2006.

(51) Int. Cl. A61K 38/18; A61P 3/04

(52) U.S. Cl. 514/12

(57) ABSTRACT

This invention relates to methods for treating unwanted body weight loss (such as cachexia), eating disorders (such as anorexia nervosa), or opioid-induced emesis by peripheral administration of a trkB agonist. The invention also relates to compositions and kits comprising a trkB agonist.

### CROSS-REFERENCE TO RELATED APPLICATIONS

This application is a continuation of U.S. patent application Ser. No. 11/670,096, which was filed on Feb. 1, 2007, which claims the benefit of U.S. Provisional Application Ser. No. 60/765,410, filed on Feb. 2, 2006, each of which is incorporated herein by reference in its entirety.

### REFERENCE TO SEQUENCE LISTING

This application is being filed electronically via EFS-Web and includes an electronically submitted sequence listing in .txt format. The .txt file contains a sequence listing entitled “PC19516BSeqList.txt” created on Jul. 21, 2009 and having a size of 3 KB. The sequence listing contained in this .txt file is part of the specification and is herein incorporated by reference in its entirety.

### FIELD OF THE INVENTION

This invention concerns use of trkB agonist in the treatment and/or prevention of unwanted weight loss, eating disorders, or opioid-induced emesis.

### BACKGROUND OF THE INVENTION

Neurotrophins are a family of small, homodimeric proteins, which play a crucial role in the development and maintenance of the nervous system. Members of the neurotrophin family include nerve growth factor (NGF), brain-derived neurotrophic factor (BDNF), neurotrophin-3 (NT-3), neurotrophin-4/5 (NT-4/5), neurotrophin-6 (NT-6), and neurotrophin-7 (NT-7). Neurotrophins, similar to other polypeptide growth factors, affect their target cells through interactions with cell surface receptors. According to current knowledge, two kinds of transmembrane glycoproteins serve as receptors for neurotrophins. Neurotrophin-responsive neurons possess a common low molecular weight (65-80 kDa), low affinity receptor (LNGFR), also known as p75NTR or p75, which binds NGF, BDNF, NT-3 and NT-4/5 with a K_{D} of 2×10⁻⁹ M; and large molecular weight (130-150 kDa), high-affinity (K_{D} in the 10⁻¹¹ M range) receptors, which are members of the trk family of receptor tyrosine kinases. The identified members of the Trk receptor family are trkA, trkB, and trkC.

Both BDNF and NT-4/5 bind to the trkB and p75NTR receptors with similar affinity. However, NT-4/5 and BDNF mutant mice exhibit quite contrasting phenotypes. Whereas NT-4/5−/− mice are viable and fertile with only a mild sensory deficit, BDNF^{-/-} mice die during early postnatal stages with severe neuronal deficits and behavioral symptoms. Fan et al., Nat. Neurosci. 3(4):350-7, 2000; Liu et al., Nature 375:238-241, 1995; Conover et al., Nature 375:235-238, 1995; Ernfors et al., Nature 368:147-150, 1994; Jones et al., Cell 76:989-999, 1994. Several publications report that NT-4/5 and BDNF have distinct biological activities in vivo and suggest that the distinct activities may result partly from differential activation of the trkB receptor and its down-stream signaling pathways by NT-4/5 and BDNF. Fan et al., Nat. Neurosci. 3(4):350-7, 2000; Minichiello et al., Neuron. 21:335-45, 1998; Wirth et al., Development. 130(23):5827-38, 2003; Lopez et al., Program No. 38.6, 2003 Abstract, Society for Neuroscience.

It has been shown that BDNF and NT-4/5 have blood glucose and blood lipid controlling activity and anti-obesity activity in type II diabetic model animals, such as C57 db/db mice. U.S. Pat. No. 6,391,312; Itakura et al., Metabolism 49:129-33 (2000); U.S. App. Pub. No. 2005/0209148; WO 2005/082401. It has also been shown that BDNF has anti-obesity activity and activity in ameliorating leptin resistance in mice fed with high fat diet. U.S. Pub. No. 2003/0036512. Kernie et al. reported that BDNF or NT-4/5 could transiently reverse the eating behavior and obesity in heterozygous BDNF knock out mice in which BDNF gene expression was reduced. Kernie et al., EMBO J. 19(6):1290-300, 2000. It has been reported that a de novo missense mutation of Y722C substitution on human trkB results in impaired receptor phosphorylation and signaling to MAP kinase; and this mutation seems to result in a unique human syndrome of hyperphagic obesity. Yeo et al., Nat. Neurosci. 7:1187-1189 (2004).

Circulating levels of BDNF in people with obesity and in patients with anorexia nervosa have been studied. Monteleone et al., Psychosomatic Medicine 66:744-748, 2004; Nakazato et al., Biol. Psychiatry 54:485-490, 2003. Contrary to the prediction based on the findings that impairments of BDNF production in mice have been associated with increased food intake, reduced energy expenditure, and weight gain, circulating BDNF is significantly reduced in the anorexia nervosa patients and significantly increased in obese subjects as compared with the non-obese healthy controls. It has been hypothesized that in anorexia nervosa, BDNF reduction, by promoting food intake, attempts to counterbalance the patients' altered behaviors that lead to a negative balance; and in obesity, increased levels of BDNF may represent an adaptive mechanism to counteract the condition of positive energy imbalance by stimulating energy expenditure and decreasing food ingestion. Monteleone et al., Psychosomatic Medicine 66:744-748, 2004.

All patents, patent applications, and publications cited herein are hereby incorporated by reference in their entirety.

### BRIEF SUMMARY OF THE INVENTION

The present invention provides methods for increasing body weight and/or food intake by peripheral administration of a trkB agonist, including a trkB selective agonist. These methods can be used for treating or preventing unwanted weight loss (such as cachexia), eating disorders (such as anorexia nervosa), and opioid-induced emesis.

In one aspect, the invention provides methods for increasing body weight in a primate comprising peripherally administering to the primate an effective amount of a trkB agonist.

In another aspect, the invention provides methods for increasing food intake in a primate comprising peripherally administering to the primate an effective amount of a trkB agonist.

In another aspect, the invention provides methods for treating or preventing cachexia in a primate comprising peripherally administering to the primate an effective amount of a trkB agonist.

In another aspect, the invention provides methods for ameliorating, reducing incidence of, or delaying the development or progression of cachexia in a primate comprising peripherally administering to the primate an effective amount of a trkB agonist.

In another aspect, the invention provides methods for treating or preventing anorexia nervosa in a primate comprising peripherally administering to the primate an effective amount of a trkB agonist.

In another aspect, the invention provides methods for ameliorating, reducing incidence of, or delaying the development or progression of anorexia nervosa in a primate comprising peripherally administering to the primate an effective amount of a trkB agonist.

In another aspect, the invention provides methods for treating or preventing opioid-induced emesis in an individual comprising peripherally administering to the individual an effective amount of a trkB agonist.

In another aspect, the invention provides methods for ameliorating, reducing incidence of, or delaying the development or progression of opioid-induced emesis in an individual comprising peripherally administering to the individual an effective amount of a trkB agonist.

The trkB agonist is administered peripherally. For example, the trkB agonist may be administered by one of the following means: intravenously, intraperitoneally, intramuscularly, subcutaneously, parenterally, via inhalation, intraarterially, intracardially, intraventricularly, and transdermally.

In some embodiments, the primate is a human. In some embodiments, the individual is a human.

The trkB agonist that can be used for the methods described herein, includes, but is not limited to, BDNF polypeptide, NT-4/5 polypeptide, and anti-trkB agonist antibodies. In some embodiments, the trkB agonist is human NT-4/5. In some embodiments, the trkB agonist is human BDNF. In other embodiments, the trkB agonist is an anti-trkB agonist antibody, including an anti-trkB agonist antibody that is trkB selective.

In another aspect, the invention provides pharmaceutical compositions comprising an effective amount of a trkB agonist, including a trkB selective agonist, and a pharmaceutically acceptable excipient. The pharmaceutical compositions may be used for treating or preventing any of the diseases described herein.

In another aspect, the invention provides kits comprising a trkB agonist, including a trkB selective antibody, for use in any of the methods described herein. In some embodiments, the kits comprise a container, a composition comprising an effective amount of a trkB agonist, in combination with a pharmaceutically acceptable excipient, and instructions for using the composition in any of the methods described herein.

In another aspect, the invention also provides methods for generating an agonist monoclonal antibody which specifically binds and activates a receptor, comprising the steps of: (a) immunizing a host mammal with an immunogenic molecule comprising an extracellular domain of the receptor by injecting the immunogenic molecule into the mammal at least two times within about 15 days. The methods may further comprise the steps of fusing lymphoic cells from the immunized mammal with an immortalized cell line to produce hybridomas that secrete monoclonal antibodies; culturing the hybridomas under the conditions that allow secretion of monoclonal antibodies; and selecting a hybridoma that secretes a monoclonal antibody that binds and activates the receptor. In some embodiments, the receptor is a receptor which requires dimerization for the activation.

### BRIEF DESCRIPTION OF THE DRAWINGS

FIG. 1 is a graph showing the effect of daily NT-4/5 infusion on body weight in obese female baboons. The X axis corresponds to days when body weight was measured and the Y axis corresponds to body weight measured as a percentage of the baseline (body weight before any treatment). Two way ANOVA was used for comparing NT-4/5 treated group and the vehicle group. Data indicated that body weight of NT-4/5 treated group was significantly different from the vehicle group (F=50.71, P<0.0001). Bonferroni posttests analysis showed significant pairwise difference between NT-4/5 treated group (solid triangles) with the vehicle group (open squares). “*” indicates P<0.05; “**” indicates P<0.01; and “***” indicates P<0.001 as indicated in the graph.

FIG. 2 is a graph showing the effect of daily NT-4/5 infusion on food intake in obese female baboons. The X axis corresponds to days when food intake was measured and the Y axis corresponds to number of biscuits taken by a baboon per day. Two way ANOVA was used for comparing NT-4/5 treated group with the vehicle group. Data indicated that food intake of NT-4/5 treated group was significantly different from the vehicle group (F=262.5, P<0.0001). Bonferroni posttests showed significant pairwise difference between NT-4/5 treated group (solid triangles) with the vehicle group (open squares). The solid black bar in the graph indicates the period when the pairwise comparison resulted in P<0.05 or less.

FIG. 3 is a graph showing the effect of twice per week NT-4/5 infusion on body weight in obese female baboons. The X axis corresponds to days when body weight was measured and the Y axis corresponds to body weight measured as a percentage of the baseline (body weight before any treatment). Two way ANOVA was used for comparing NT-4/5 treated group with the vehicle group. Data indicated that body weight of NT-4/5 treated group is significantly different from the vehicle group (F=34.81, P<0.0001). Bonferroni posttests analysis showed significant pairwise difference between NT-4/5 treated group (solid triangles) with the vehicle group (open squares). “*” indicates P<0.05; and “**” indicates P<0.01.

FIG. 4 is a graph showing the effect of twice per week NT-4/5 infusion on food intake in obese female baboons. The X axis corresponds to days when food intake was measured and the Y axis corresponds to number of biscuits taken by a baboon per day.

FIG. 5 is a graph showing the effect of daily NT-4/5 and weekly pegylated NT-4/5 infusion on body weight in lean cynomolgus monkeys. The X axis corresponds to days when body weight was measured and the Y axis corresponds to body weight measured as a percentage of the baseline (body weight before any treatment). Two way ANOVA was used for comparing NT-4/5 treated group or the pegylated NT-4/5 (PEG-NT-4/5) with the vehicle group. Data indicated that body weight of NT-4/5 treated group, but not pegylated NT-4/5 treated group, was significantly different from the vehicle group (F=54.98, P<0.0001). Bonferroni posttests analysis showed significant pairwise difference between NT-4/5 treated group (triangles) and the vehicle group (squares), but not between the pegylated NT-4/5 group (inverted triangles) and the vehicle group. “***” indicates P<0.001 as indicated in the graph.

FIG. 6 is a graph showing the effect of daily NT-4/5 and weekly pegylated NT-4/5 infusion on food intake in lean cynomolgus monkeys. The X axis corresponds to days when food intake was measured and the Y axis corresponds to number of biscuits taken by a monkey per day. Two way ANOVA was used for comparing NT-4/5 treated group or the pegylated NT-4/5 (PEG-NT-4/5) with the vehicle group. Data indicated that body weight of NT-4/5 treated group, but not the pegylated NT-4/5 treated group, was significantly different from the vehicle group (F=33.82, P<0.0001). Bonferroni posttests showed significant pairwise difference (P<0.05 or less) between NT-4/5 treated group (triangles) and the vehicle group (squares) on day 15, 16, 17, 19, 22, 23, 25, and 30, but no significant pairwise difference between the pegylated NT-4/5 group (inverted triangles) and the vehicle group.

FIG. 7 is a graph showing the effect of daily NT-4/5 and daily pegylated NT-4/5 subcutaneous injection on body weight in lean cynomolgus monkeys. The X axis corresponds to days when body weight was measured and the Y axis corresponds to body weight measured as a percentage of the baseline (body weight before any treatment). Two way ANOVA was used for comparing NT-4/5 treated group or the pegylated NT-4/5 (PEG-NT-4/5) with the vehicle group. Data indicated that body weight of NT-4/5 treated group was significantly different from the vehicle group (F=19.10, P<0.0001). Bonferroni posttests analysis showed significant pairwise difference between NT-4/5 treated group (triangles) with the vehicle group (squares), and between the pegylated NT-4/5 group (inverted triangles) and the vehicle group. “***” indicates P<0.001; and “**” indicates P<0.01.

FIG. 8 is a graph showing effect of single injection of NT-4/5 on morphine-induced emesis in ferrets. The X axis corresponds to type of drug injected; and the Y axis corresponds to number of retches and vomits over a period of 60 min post injection. One way ANOVA with Dunnett's posttest was used for statistical analysis. P values are indicated in the graph.

FIG. 9A and FIG. 9B show the induction of c-Fos in ferret hindbrain by NT-4/5. FIG. 9A shows number of nuclei that are stained by anti-c-Fos antibody in the area postrema. FIG. 9B shows number of nuclei that are stained by anti-c-Fos antibody in the dorsal vagal nucleus.

FIG. 10 shows level of trkB tyrosine phosphorylation in KIRA assay by various anti-trkB antibodies (36D1, 38B8, 37D12, 19H8(1), 1F8, 23B8, 18H6) in comparison to human NT-4/5.

FIG. 11 shows a graph of the nodose neuron survival supported by several trkB agonist antibodies. The X-axis represents the different concentrations of anti-trkB antibodies added to the embryonic day 15 (E15) nodose neuron culture obtained from Swiss Webster mice. The Y-axis represents the number of surviving neurons 48 hours post plating. Each point is an average of four determinations and the error bars show variance from that average of one standard deviation. The data indicate that some of the trkB antibodies tested can support nodose neuron survival and that the 50% effective concentration (EC50) of these antibodies under this culture condition range from less than 0.1 to over 10 pM (See table 1).

FIG. 12A and FIG. 12B are graphs showing the effect of intracranial injections of anti-trkB agonist antibodies on body weight (FIG. 12A) and food intake (FIG. 12B) in mice. Antibodies and NT-4/5 were injected on day 0. Body weight and food intake were measured daily until day 15. “***” indicates P<0.001 as compared to mouse IgG control; “**” indicates P<0.01 as compared to mouse IgG control; and “*” indicates P<0.05 as compared to mouse IgG control.

FIG. 13A and FIG. 13B are graphs showing the effect of peripheral intravenous injections of anti-trkB agonist antibody on body weight (FIG. 13A) and food intake (FIG. 13B) in cynomolgus monkeys. Antibodies were injected on day 1. Body weight was monitored weekly and food intake was monitored daily. “***” indicated P>0.001 as compared to control vehicle; “**” indicates P>0.01 as compared to control vehicle; and “*” indicates P>0.05 as compared to control vehicle.

### DETAILED DESCRIPTION OF THE INVENTION

The present invention provides methods for treating or preventing unwanted weight loss (such as cachexia), eating disorders (such as anorexia nervosa), and opioid-induced emesis comprising administering a trkB agonist to an individual.

### I. GENERAL TECHNIQUES

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology. A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### II. DEFINITIONS

As used herein, “treatment” is an approach for obtaining beneficial or desired clinical results. For purposes of this invention, beneficial or desired clinical results include, but are not limited to, one or more of the following: improving, lessening severity, alleviation of one or more symptoms associated with a disease. For example, for treatment of cachexia, beneficial or desired clinical results include, but are not limited to, any improvement, lessening of severity, and/or alleviation of any one or more of the following: weight loss, lipolysis, loss of muscle and visceral protein, anorexia (i.e., loss of appetite), reduced food/caloric intake, chronic nausea, fatigue and weakness. For treatment of anorexia nervosa, beneficial or desired clinical results include, but are not limited to, any one or more of the following: improvement of appetite, attenuation of food resentment, gaining weight, maintaining normal nutritional status, hydration and electrolyte balance, maintaining normal body weight for age and height, reducing frequency and duration of hospitalization, and reducing risk of death. For treatment of opioid-induced emesis, beneficial or desired clinical results include, but are not limited to, lessening the severity and/or shortening the duration of nausea and/or vomiting, thereby allowing the full clinical benefits of opioid-induced pain relief.

“Ameliorating” a disease or one or more symptoms of the disease means a lessening or improvement of one or more symptoms associated with the disease as compared to not administering a trkB agonist. “Ameliorating” also includes shortening or reduction in duration of a symptom.

“Reducing incidence” of a disease means any of reducing severity (which can include reducing need for and/or amount of (e.g., exposure to) other drugs and/or therapies generally used for this condition), duration, and/or frequency (including, for example, delaying or increasing time to next episodic attack in an individual). As is understood by those skilled in the art, individuals may vary in terms of their response to treatment, and, as such, for example, a method of reducing incidence of a disease in an individual reflects administering the trkB agonist based on a reasonable expectation that such administration may likely cause such a reduction in incidence in that particular individual.

As used therein, “delaying” the development of a disease means to defer, hinder, slow, retard, stabilize, and/or postpone progression of the disease. This delay can be of varying lengths of time, depending on the history of the disease and/or individuals being treated. As is evident to one skilled in the art, a sufficient or significant delay can, in effect, encompass prevention, in that the individual does not develop the disease (e.g., cachexia, anorexia nervosa, and opioid-induced emesis). A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Development” or “progression” of a disease means initial manifestations and/or ensuing progression of the disorder. Development of a disease can be detectable and assessed using standard clinical techniques well known in the art. However, development also refers to progression that may be undetectable. For purpose of this invention, development or progression refers to the biological course of the symptoms. “Development” includes occurrence, recurrence, and onset. As used herein “onset” or “occurrence” of a disease includes initial onset and/or recurrence.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing intensity, duration, or frequency of attack of the disease, and decreasing one or more symptoms resulting from the disease (biochemical, histological and/or behavioral), including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual” or a “subject” is a mammal, more preferably, a human. Mammals also include, but are not limited to, farm animals, sport animals, pets, primates (including humans), horses, dogs, cats, mice and rats.

An “trkB agonist” refers to an agent that is able to bind to and activate a trkB receptor and/or downstream pathway(s) mediated by the trkB signaling function. For example, the agonist may bind to the extracellular domain of a trkB receptor and thereby cause dimerization of the receptor, resulting in activation of the intracellular catalytic kinase domain. Consequently, this may result in stimulation of growth and/or differentiation of cells expressing the receptor in vitro and/or in vivo. In some embodiments, a trkB agonist binds to trkB and activates a trkB biological activity.

“Biological activity”, when used in conjunction with the trkB agonist of the present invention, generally refers to having the ability to bind and activate the trkB receptor and/or a downstream pathway mediated by the trkB signaling function. As used herein, “biological activity” encompasses one or more effector functions in common with those induced by action of NT-4/5 and/or BDNF, the native ligand of trkB, on a trkB-expressing cell. Without limitation, biological activities include any one or more of the following: ability to bind and activate trkB; ability to promote trkB receptor dimerization; the ability to promote the development, survival, function, maintenance and/or regeneration of cells (including damaged cells), in particular neurons in vitro or in vivo, including peripheral (sympathetic, sensory, motor, and enteric) neurons, and central (brain and spinal cord) neurons, and non-neuronal cells, e.g. peripheral blood leukocytes, endothelial cells and vascular smooth muscle cells. A particular preferred biological activity is the ability to increase body weight and/or food intake in a primate when administered peripherally, to treat (including prevention of) one or more symptoms of cachexia and anorexia nervosa in a primate, and/or to treat (including prevention of) one or more symptoms of opioid-induced emesis in a mammal.

An “agonist anti-trkB antibody” (interchangeably termed “anti-trkB agonist antibody”) refers to an antibody that is able to bind to and activate a trkB receptor and/or downstream pathway(s) mediated by the trkB signaling function. For example, the agonist antibody may bind to the extracellular domain of a trkB receptor and thereby cause dimerization of the receptor, resulting in activation of the intracellular catalytic kinase domain. Consequently, this may result in stimulation of growth and/or differentiation of cells expressing the receptor in vitro and/or in vivo. In some embodiments, an agonist anti-trkB antibody binds to trkB and activates a trkB biological activity.

As used herein, “peripheral administration” or “administered peripherally” refers to introducing an agent into a subject outside of the central nervous system (CNS) or blood brain barrier (BBB). Peripheral administration encompasses any route of administration other than direct administration to the spine or brain. Peripheral administration can be local or systemic.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion (such as domain antibodies), and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

As used herein, “humanized” antibodies refer to forms of non-human (e.g. murine) antibodies that are specific chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

An epitope that “preferentially binds” or “specifically binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a trkB epitope is an antibody that binds this epitope with greater affinity, avidity, more readily, and/or with greater duration than it binds to other trkB epitopes or non-trkB epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3.

As used herein, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, FcγRIII, and FcγRIV subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; de Haas et al., 1995, J. Lab. Clin. Med., 126:330-41; Nimmerjahn et al., 2005, Immunity 23:2-4. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202:163 (1996), may be performed.

A “functional Fc region” possesses at least one effector function of a native sequence Fc region. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably, at least about 95%, at least about 96%, at least about 97%, at least about 98%, at least about 99% sequence identity therewith.

As used herein “antibody-dependent cell-mediated cytotoxicity” and “ADCC” refer to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in a animal model such as that disclosed in Clynes et al., 1998, PNAS (USA), 95:652-656.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, -anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “substantially pure” refers to material which is at least 50% pure (i.e., free from contaminants), more preferably, at least 90% pure, more preferably, at least 95% pure, more preferably, at least 98% pure, more preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

The term “kₒₙ”, as used herein, is intended to refer to the rate constant for association of an antibody to an antigen.

The term “k_{off}”, as used herein, is intended to refer to the rate constant for dissociation of an antibody from the antibody/antigen complex.

The term “K_{D}”, as used herein, is intended to refer to the equilibrium dissociation constant of an antibody-antigen interaction.

As used herein, the singular form “a”, “an”, and “the” includes plural references unless indicated otherwise.

### III. METHODS OF THE INVENTION

The present invention encompasses methods for increasing body weight and/or food intake by peripheral administration of a trkB agonist. These methods can be used for treating or preventing unwanted weight loss (such as cachexia) and eating disorders (such as anorexia nervosa) in primates, and opioid-induced emesis in mammals. The methods entail peripheral administration of an effective amount of one or more trkB agonists to an individual in need thereof (various indications and aspects are described herein).

With respect to all methods described herein, reference to trkB agonists also include compositions comprising one or more of these agents. These compositions may further comprise suitable excipients, such as pharmaceutically acceptable excipients including buffers, which are well known in the art. The present invention can be used alone or in combination with other conventional methods of treatment.

Cachexia that can be treated and/or prevented by the methods described herein may be caused and/or associated with one or more of the following: chronic obstructive pulmonary disease (COPD), chronic kidney disease (CKD), chronic heart failure (CHF), aging, cancer, and AIDS. In some embodiments, the human patients having cachexia treated have a Body Mass Index (BMI, calculated as body weight per height in meters squared (kg/m²)) less than about any of 25.0 kg/m², 24.0 kg/m², 23.0 kg/m², 22.0 kg/m², 21.0 kg/m², 20.0 kg/m², 19.0 kg/m², and 18.5 kg/m². In some embodiments, the human patients having cachexia treated have a daily food intake less than about 90%, about 80%, about 70%, about 60%, about 50%, about 40%, about 30%, about 20%, or about 10% of the normal recommended daily intake level or pre-morbid level.

In some embodiments, the human patients having anorexia nervosa treated by the methods described herein have a BMI less than any of about 18.5 kg/m², 17.5 kg/m², and 16.5 kg/m². In some embodiments, the human patients having anorexia nervosa treated have a daily food intake less than about 90%, about 80%, about 70%, about 60%, about 50%, about 40%, about 30%, about 20%, or about 10% of the normal recommended daily intake level or pre-morbid level.

The trkB agonist is administered peripherally. It is understood that although the agent is administered peripherally, a small percentage of the agent may pass blood brain barrier and result in delivery to the central nervous system depending on the properties of the agent. In some embodiments, less than any of about 1%, about 0.5%, about 0.25%, and about 0.1% of peripherally administered trkB agonist (for example, trkB agonist antibody) gains access to the CNS.

The trkB agonist can be administered to an individual via any suitable peripheral route. It should be apparent to a person skilled in the art that the examples described herein are not intended to be limiting but to be illustrative of the techniques available. Accordingly, in some embodiments, the trkB agonist is administered to an individual in accord with known methods, such as intravenous administration, e.g., as a bolus or by continuous infusion over a period of time, by intramuscular, intraperitoneal, subcutaneous, intra-articular, sublingually, intrasynovial, via insufflation, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively, trkB agonist can be aerosolized using a fluorocarbon formulation and a metered dose inhaler, or inhaled as a lyophilized and milled powder.

A trkB agonist may be administered via site-specific or targeted local delivery techniques outside of the CNS or the blood brain barrier. Examples of site-specific or targeted local delivery techniques include various implantable depot sources of the trkB agonist or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of trkB agonists may be used for administration. In some embodiments, a trkB agonist may be administered neat. In other embodiments, a trkB agonist and a pharmaceutically acceptable excipient are administered, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000). Generally, these agents are formulated for administration by injection (e.g., intraperitoneally, intravenously, subcutaneously, intramuscularly, etc.), although other forms of administration (e.g., oral, mucosal, transdermal, inhalation, etc) can be also used.

The particular dosage regimen, i.e., dose, timing and repetition, will depend on the particular individual and that individual's medical history, the particular disease (e.g., cachexia, anorexia nervosa, and opioid-induced emesis) to be treated, and the particular trkB agonist. Generally, any of the following doses of trkB agonist (e.g., NT-4/5, BDNF, and anti-trkB agonist antibody) may be used: a dose of at least about 50 mg/kg body weight; at least about 20 mg/kg body weight; at least about 10 mg/kg body weight; at least about 5 mg/kg body weight; at least about 3 mg/kg body weight; at least about 2 mg/kg body weight; at least about 1 mg/kg body weight; at least about 750 μg/kg body weight; at least about 500 μg/kg body weight; at least about 250 ug/kg body weight; at least about 100 μg/kg body weight; at least about 50 μg/kg body weight; at least about 10 ug/kg body weight; at least about 1 μg/kg body weight or more, is administered. Empirical considerations, such as the half-life, generally will contribute to determination of the dosage. For repeated administrations over several days or longer, depending on the condition, the treatment is sustained until a desired suppression of disease symptoms occurs or until sufficient therapeutic levels are achieved. For example, dosing from one to five times a week is contemplated. Other dosing regimens include a regimen of up to 1 time per day, 1 to 5 times per week, or less frequently. In some embodiments, the trkB agonist is administered about once per week, about 1 to 4 times per month. Intermittent dosing regime with staggered dosages spaced by 2 days up to 7 days or even 14 days may be used. In some embodiments, treatment may start with a daily dosing and later change to weekly even monthly dosing. The progress of this therapy is easily monitored by conventional techniques and assays.

In some individuals, more than one dose may be required. Frequency of administration may be determined and adjusted over the course of therapy. For example, frequency of administration may be determined or adjusted based on the type and severity of the disease to be treated, whether the agent is administered for preventive or therapeutic purposes, previous therapy, the patient's clinical history and response to the agent, and the discretion of the attending physician. Typically the clinician will administer a trkB agonist until a dosage is reached that achieves the desired result. In some cases, sustained continuous release formulations of trkB agonist may be appropriate. Various formulations and devices for achieving sustained release are known in the art. For example, trkB agonist may be administered through a mechanical pump or embedded in a matrix bed for sustained or slow release.

In one embodiment, dosages for trkB agonist may be determined empirically in individuals who have been given one or more administration(s). Individuals are given incremental dosages of trkB agonist. To assess efficacy of trkB agonist, markers of the disease state can be monitored. It will be apparent to one of skill in the art that the dosage will vary depending on the individual, the stage of the disease (such as cachexia, anorexia nervosa, and opioid-induced emesis), and the past and concurrent treatments being used.

Administration of trkB agonist in accordance with the method in the present invention can be continuous or intermittent, depending, for example, upon the recipient's physiological condition, whether the purpose of the administration is therapeutic or prophylactic, and other factors known to skilled practitioners. The administration of an trkB agonist may be essentially continuous over a preselected period of time or may be in a series of spaced doses.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

Assessment of disease is performed using standard methods known in the arts, for example, by monitoring appropriate marker(s). For example, for cachexia, the following markers may be monitored: body weight, plasma albumin, body fat, body lean mass, fatigue, weakness, and appetite. For anorexia nervosa, the following markers may be monitored: body weight, appetite, and fear of gaining weight. For opioid-induced emesis, the following markers may be monitored: nausea, vomiting, appetite, body weight, and other associated medical complications.

### IV. COMPOSITIONS AND METHODS OF MAKING THE COMPOSITIONS

The methods of the invention use a trkB agonist, which refers to any molecule that binds and activates a native trkB receptor and/or downstream pathways mediated by the trkB signaling function. The trkB agonist includes any native ligand of a trkB receptor, such as NT-4/5 and BDNF. The trkB agonist also includes non-native ligand (e.g., polypeptides, peptide-derived compound, cyclic peptide-derived or non-peptide derived molecules) of a trkB receptor that binds to and activates a native trkB receptor, thereby mimicking a biological activity of a native ligand of the receptor. An example of non-native ligands of a trkB receptor is a anti-trkB agonist antibody. TrkB agonists also include small molecules or peptide mimetics (e.g., peptide mimetics of BDNF). See, e.g., O'Leary et al., J. Biol. Chem. 278:25738-44, 2003. In some embodiments, the small molecule trkB agonist does not significantly pass blood brain barrier when administered peripherally.

A trkB agonist should exhibit any one or more of the following characteristics: (a) bind to trkB receptor; (b) bind to trkB receptor and activate trkB biological activity(ies) and/or one or more downstream pathways mediated by trkB signaling function(s); (c) bind to trkB receptor and increase body weight and/or food intake in a primate when administered peripherally; (d) bind to trkB receptor and treat, prevent, reverse, or ameliorate one or more symptoms of cachexia in a primate when administered peripherally; (e) bind to trkB receptor and treat, prevent, reverse, or ameliorate one or more symptoms of anorexia nervosa in a primate when administered peripherally; (f) bind to trkB receptor and treat, prevent, reverse, or ameliorate one or more symptoms of opioid-induced emesis in a mammal when administered peripherally; (g) promote trkB receptor dimerization and activation; and (h) increase trkB receptor-dependent neuronal survival and/or neurite outgrowth. In some embodiments, the trkB binds and activates trkB receptor, but does not significantly or preferentially activate one or more other trk receptors, such as trkA and/or trkC.

The trkB agonist may be in the form of a composition for use in any of the methods described herein. The composition used in the methods of the invention comprises an effective amount of a trkB agonist. The composition can further comprise pharmaceutically acceptable carriers, excipients, or stabilizers (Remington: The Science and practice of Pharmacy 20th Ed. (2000) Lippincott Williams and Wilkins, Ed. K. E. Hoover.), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

TrkB agonists described herein can be formulated for sustained-release. Suitable examples of sustained-release preparations include semipermeable matrices of solid hydrophobic polymers containing trkB agonist, which matrices are in the form of shaped articles, e.g. films, or microcapsules. Examples of sustained-release matrices include polyesters, hydrogels (for example, poly(2-hydroxyethyl-methacrylate), or ‘poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid. Another example of sustained release drug-delivery system that can be used is the ATRIGEL® made by Atrix Laboratories. See, for example U.S. Pat. No. 6,565,874. The ATRIGEL® drug delivery system consists of biodegradable polymers, similar to those used in biodegradable sutures, dissolved in biocompatible carriers. TrkB agonists may be blended into this liquid delivery system at the time of manufacturing or, depending upon the product, may be added later by the physician at the time of use. When the liquid product is injected subcutaneously or intramuscularly through a small gauge needle or placed into accessible tissue sites through a cannula, displacement of the carrier with water in the tissue fluids causes the polymer to precipitate to form a solid film or implant. TrkB agonists encapsulated within the implant are then released in a controlled manner as the polymer matrix biodegrades with time. Depending upon the patient's medical needs, the Atrigel system can deliver proteins over a period ranging from days to months. Injectable sustained release systems, such as ProLease®, Medisorb®, manufactured by Alkermes may also be used.

In some embodiments, the invention provides compositions (described herein) for use in any of the methods described herein, whether in the context of use as a medicament and/or use for manufacture of a medicament.

NT-4/5 Polypeptides

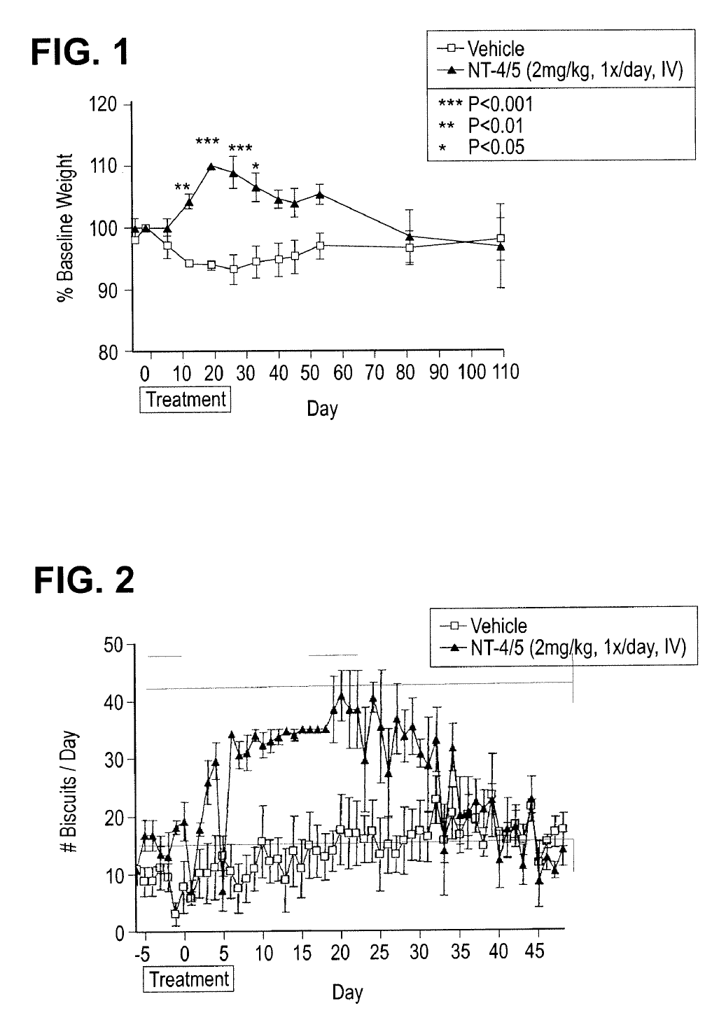

The trkB agonist used in the methods of the invention includes NT-4/5 polypeptides. As used herein, “NT-4/5 polypeptide” includes naturally-occurring mature protein (interchangeably termed “NT-⅘”) such as mature human NT-4/5 shown in Table 1 below, and in U.S. Pat. Appl. Pub. No. 2005/0209148 and PCT WO 2005/08240 and FIG. 1 in U.S. Pat. Appl. Pub. No. 20030203383 and naturally occurring amino acid sequence variants of NT-4/5; amino acid sequence variants of NT-4/5; peptide fragments of mature NT-4/5 (such as human) and said amino acid sequence variants; and modified forms of mature NT-4/5 and said amino acid sequence variants and peptide fragments wherein the polypeptide or peptide has been covalently modified by substitution with a moiety other than a naturally occurring amino acid, as long as the amino acid sequence variant, peptide fragment, and the modified form thereof show one or more biological activities of a trkB agonist and/or of naturally occurring mature NT-4/5 protein. The trkB agonist also includes fusion proteins and conjugates comprising any of the NT-4/5 polypeptide embodiments described herein, e.g., an NT-4/5 polypeptide conjugated or fused to a half life extending moiety, such as a PEG, IgG Fc region, albumin, or a peptide. The amino acid sequence variants, peptide fragments (including fragments of variants), or modified forms thereof under consideration do not include NGF, BDNF, or NT-3 of any animal species. Variants, peptide fragments, and modified forms of naturally occurring NT-4/5 are described in U.S. Pat. Appl. Pub. Nos. 2003/0203383; 2002/0045576; 2005/0209148; U.S. Pat. Nos. 5,702,906; 6,506,728; 6,566,091; 5,830,858; which are incorporated by reference in their entirety. NT-4/5 polypeptides include any one or more embodiments described herein. For example, NT-4/5 polypeptide comprises a naturally occurring sequence with one or more amino acid insertion, deletion, or substitution.

TABLE 1

Amino acid sequence of mature human NT-4/5
and the human nucleotide sequence encoding
the mature human NT-4/5

Amino acid sequence (SEQ ID NO: 1):
GVSETAPASRRGELAVCDAVSGWVTDRRTAVDLRGREVEVLGEVPAAGGS
PLRQYFFETRCKADNAEEGGPGAGGGGCRGVDRRHWVSECKAKQSYVRAL
TADAQGRVGWRWIRIDTACVCTLLSRTGRA
Nucleotide sequence (SEQ ID NO: 2)
GGGGTGAGCGAAACTGCACCAGCGAGTCGTCGGGGTGAGCTGGCTGTGTG
CGATGCAGTCAGTGGCTGGGTGACAGACCGCCGGACCGCTGTGGACTTGC
GTGGGCGCGAGGTGGAGGTGTTGGGCGAGGTGCCTGCAGCTGGCGGCAGT
CCCCTCCGCCAGTACTTCTTTGAAACCCGCTGCAAGGCTGATAACGCTGA
GGAAGGTGGCCCGGGGGCAGGTGGAGGGGGCTGCCGGGGAGTGGACAGGA
GGCACTGGGTATCTGAGTGCAAGGCCAAGCAGTCCTATGTGCGGGCATTG
ACCGCTGATGCCCAGGGCCGTGTGGGCTGGCGATGGATTCGAATTGACAC
TGCCTGCGTCTGCACACTCCTCAGCCGGACTGGCCGGGCCTGAG

In some embodiments, the NT-4/5 polypeptide is a mammalian NT-4/5 polypeptide which may be a naturally occurring mammalian NT-4/5, or NT-4/5 polypeptide derived from a naturally occurring mammalian NT-4/5 and having a sequence that does not match any part of a naturally occurring non-mammalian NT-4/5. In some embodiments, the NT-4/5 polypeptide is a human NT-4/5 polypeptide which may be a naturally occurring human NT-4/5, or NT-4/5 polypeptide derived from a naturally occurring human NT-4/5 and having a sequence that does not match any part of a naturally occurring non-human NT-4/5.

NT-4/5 polypeptides, including variants, peptide fragments, modified forms of NT-4/5 polypeptides (including naturally occurring NT-4/5), fusion protein and conjugate of the invention are characterized by any (one or more) of the following characteristics: (a) bind to trkB receptor; (b) bind to trkB receptor and activate trkB biological activity(ies) and/or one or more downstream pathways mediated by trkB signaling function(s); (c) bind to trkB receptor and increase body weight and/or food intake in a primate when administered peripherally; (d) bind to trkB receptor and treat, prevent, reverse, or ameliorate one or more symptoms of cachexia in a primate when administered peripherally; (e) bind to trkB receptor and treat, prevent, reverse, or ameliorate one or more symptoms of anorexia nervosa in a primate when administered peripherally; (f) bind to trkB receptor and treat, prevent, reverse, or ameliorate one or more symptoms of opioid-induced emesis in a mammal when administered peripherally; (g) promote trkB receptor dimerization and activation; and (h) increase trkB receptor-dependent neuronal survival and/or neurite outgrowth. Thus all NT-4/5 polypeptides (including variants, fragments, and modified forms) are functional as described above.

Biological activity of variants may be tested in vitro and in vivo using methods known in the art and methods described herein. Methods described herein for identifying anti-trkB agonist may also be used. NT-4/5 polypeptides may have an enhanced activity or reduced activity as compared to a naturally occurring NT-4/5 protein. In some embodiments, functionally equivalent variants have at least about any of 50%, 60%, 70%, 75%, 80%, 85%, 90%, or 95% of activity as compared to the native NT-4/5 protein from which the NT-4/5 polypeptide is derived with respect to one or more of the biological assays described above (or known in the art). In some embodiments, functionally equivalent variants have an EC₅₀ (half of the maximal effective concentration) of less than about any of 0.01 nM, 0.1 nM, 1 nM, 10 nM, or 100 nM in TrkB receptor activation in vitro (e.g., assays described in Example 6, and in US 2005/0209148 and PCT WO 2005/082401).

Amino acid sequence variants of NT-4/5 include polypeptides having an amino acid sequence which differs from naturally occurring NT-4/5 by virtue of the insertion, deletion, and/or substitution of one or more amino acid residues within the sequence of naturally occurring NT-4/5 (for example, mature human NT-4 shown in Table 1). Amino acid sequence variants generally will be at least about 65%, 70%, 75%, 80%, 85%, 90%, 95%, 96%, 97%, 98%, or 99% identical to any naturally occurring NT-4/5 (such as mature human NT-4/5 shown in SEQ ID NO:1). In some embodiments, the variant is at least about 70% identical to the amino acid sequence of SEQ ID NO:1. In some embodiments, the variant is at least about 85% identical to the amino acid sequence of SEQ ID NO:1. In some embodiments, the variant is at least about 90% identical to the amino acid sequence of SEQ ID NO:1. In some embodiments, the variant is at least about 95% identical to the amino acid sequence of SEQ ID NO:1.

Amino acid sequence variants of NT-4/5 can be generated by making predetermined mutations in a previously isolated NT-4/5 DNA. Amino acid variants may be designed and generated based on crystal structure of NT-4/5 and TrkB receptor. Banfield et al., Structure 9: 1191-9 (2001) For example, amino acids that are not directly involved in interaction between monomers of NT-4/5 and between NT-4/5 and the TrkB receptor may be mutated, for example, to introduce PEG attaching site. Methods known in the art may be used to design variants of NT-4/5 polypeptide that have enhanced or reduced one or more biological activities as compared to the naturally occurring NT-4/5 protein.

There are two principal variables to consider in making such predetermined mutations: the location of the mutation site and the nature of the mutation. In general, the location and nature of the mutation chosen generally depends upon the NT-4/5 characteristic to be modified. For example, candidate NT-4/5 antagonists or super agonists initially can be selected by locating amino acid residues that are identical or highly conserved among NGF, BDNF, NT-3, and NT-4. Those residues can then be modified in series, e.g., by (1) substituting first with conservative choices and then with more radical selections depending upon the results achieved, (2) deleting the target residue, or (3) inserting residues of the same or different class adjacent to the located site, or combinations of options 1-3.

One helpful technique is called “ala scanning”. Here, an amino acid residue or group of target residues are identified and substituted by alanine or polyalanine. Those domains demonstrating functional sensitivity to the alanine substitutions then are refined by introducing further or other variants at or for the sites of alanine substitution.

Obviously, such variations which, for example, convert NT-4/5 into NGF, BDNF, or NT-3 are not included within the scope of this invention. Thus, while the site for introducing an amino acid sequence variation is predetermined, the nature of the mutation per se need not be predetermined. For example, to optimize the performance of a mutation at a given site, ala scanning or random mutagenesis is conducted at the target codon or region and the expressed NT-4/5 variants are screened for the optimal desired activity.

Amino acid sequence deletions generally range from about 1 to 30 residues, more preferably, about 1 to 10 residues, and typically are contiguous. Deletions may be introduced into regions of low homology among BDNF, NGF, NT-3, and NT-4/5 to modify the activity of NT-4/5. Deletions from NT-4/5 in areas of substantial homology with BDNF, NT-3, and NGF may be more likely to modify the biological activity of NT-4/5 more significantly. The number of consecutive deletions may be selected so as to preserve the tertiary structure of NT-4/5 in the affected domain, e.g., beta-pleated sheet or alpha helix.

Amino acid sequence insertions include amino- and/or carboxyl-terminal fusions ranging in length from one residue to polypeptides containing a thousand or more residues, as well as intrasequence insertions of single or multiple amino acid residues. Intrasequence insertions (i.e., insertions within the mature NT-4/5 sequence) may range generally from about 1 to 10 residues, more preferably, 1 to 5, most preferably 1 to 3. An example of a terminal insertion includes fusion of a heterologous N-terminal signal sequence to the N-terminus of the NT-4/5 molecule to facilitate the secretion of mature NT-4/5 from recombinant host. Such signals generally will be homologous to the intended host cell and include STII or Ipp for E. coli, alpha factor for yeast, and viral signals such as herpes gD for mammalian cells. Other insertions include the fusion of a polypeptide to the N- or C-termini of NT-4/5.

Another group of variants includes those in which at least one amino acid residue in NT-4/5, and preferably only one, has been removed and a different residue inserted in its place. An example is the replacement of arginine and lysine by other amino acids to render the NT-4/5 resistant to proteolysis by serine proteases, thereby creating a variant of NT-4/5 that is more stable. The sites of greatest interest for substitutional mutagenesis include sites where the amino acids found in BDNF, NGF, NT-3, and NT-4 are substantially different in terms of side chain bulk, charge or hydrophobicity, but where there also is a high degree of homology at the selected site within various animal analogues of NGF, NT-3, and BDNF (e.g. among all the animal NGFs, all the animal NT-3, and all the BDNFs). This analysis will highlight residues that may be involved in the differentiation of activity of the trophic factors, and therefore, variants at these sites may affect such activities. Examples of such sites in mature human NT-4/5, numbered from the N-terminal end, and exemplary substitutions include G77 to K, H, Q or R and R84 to E, F, P, Y or W of NT-4/5 of SEQ ID NO:1, respectively. Other sites of interest are those in which the residues are identical among all animal species BDNF, NGF, NT-3, and NT-4/5, this degree of conformation suggesting importance in achieving biological activity common to all four factors.

For example, substitution of one or more amino acids includes conservative substitutions. Methods of making conservative substitutions are known in the art. For example, ala (A) may be substituted by val, leu, ile, preferably by val; arg (R) may be substituted by lys, gin, asn, preferably by lys; asn (N) may be substituted by gin, his, lys, arg, preferably by gin; asp (D) may be substituted by glu; cys (C) may be substituted by ser; gin (O) may be substituted by asn; glu (E) may be substituted by asp; gly (G) may be substituted by pro; his (H) may be substituted by asn, gin, lys, arg; preferably by arg; ile (I) may be substituted by leu, val, met, ala, phe, norleucine, preferably by leu; leu (L) may be substituted by norleucine, ile, val, met; ala; phe, preferably by ile; lys (K) may be substituted by arg; gin, asn, preferably by arg; met (M) may be substituted by leu; phe; ile, preferably by leu; phe (F) may be substituted by leu, val, ile, ala, preferably by leu; pro (P) may be substituted by gly; ser (S) may be substituted by thr; thr (T) may be substituted by ser; trp (W) may be substituted by tyr; tyr (Y) may be substituted by trp, phe, thr, ser, preferably by phe; val (V) may be substituted by ile; leu; met; phe, ala; norleucine, preferably by leu.

Sites particularly suited for conservative substitutions include, numbered from the N-terminus of the mature human NT-4 (SEQ ID NO:1), R11, G12, E13, V16, D18, W23, V24, D26, V40, L41, Q54, Y55, F56, E58, T59, G77, R79, G80, H85, W86, A99, L100, T101, W110, R111, W112, I113, R114, I115, D116, and A118. Cysteine residues not involved in maintaining the proper conformation of NT-4/5 also may be substituted, generally with serine, in order to improve the oxidative stability of the molecule and prevent aberrant crosslinking. Sites other than those set forth in this paragraph are suitable for deletional or insertional studies generally described above.

Substantial modifications in function may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side chain properties (some of these may fall into several functional groups):
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another.

Examples of NT-4 variants include: polypeptide of SEQ ID NO:1 with mutation of E67 to S or T (this adds an N-linked glycosylation site); polypeptide from amino acid residue R83 to Q94, G1 to C61, G1 to C17, C17 to C61, C17 to C78, C17 to C90, C17 to C119, C17 to C121, R11 to R27, R11 to R34, R34 to R53, C61 to C78, R53 to C61, C61 to C119, C61 to C78, C78 to C119, C61 to C90, R60 to C78, K62 to C119, K62 to K91, R79 to R98, R83 to K93, T101 to R111, G1 to C121 of SEQ ID NO:1; polypeptide comprises V40-C121 of SEQ ID NO:1, for example, V40-C121 of SEQ ID NO:1 fused to a polypeptide at the N-terminal and/or C-terminal; polypeptide comprises SEQ ID NO:1 with deletion of C78, C61, Q54-T59, R60-D82, H85-S88, W86-T101 (deletions of the indicated span of residues, inclusive); SEQ ID NO:1 with mutation from R53 to H, from K91 to H, from V108 to F, from R84 to Q, H, N, T, Y or W, and from D116 to E, N, Q, Y, S or T. Also included is NT-4/5 (SEQ ID NO:1) wherein position 70 is substituted with an amino acid residue other than G, E, D or P; position 71 with other than A, P or M; and/or position 83 with other than R, D, S or K; as well as cyclized NT-4 fragments.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Amino acid sequence variants of NT-4/5 may be naturally occurring or may be prepared synthetically, such as by introducing appropriate nucleotide changes into a previously isolated NT-4/5 DNA, or by in vitro synthesis of the desired variant polypeptide. As indicated above, such variants may comprise deletions from, or insertions or substitutions of, one or more amino acid residues within the amino acid sequence of mature NT-4/5 (e.g., sequence shown in Table 1). Any combination of deletion, insertion, and substitution is made to arrive at an amino acid sequence variant of NT-4/5, provided that the resulting variant polypeptide possesses a desired characteristic. The amino acid changes also may result in further modifications of NT-4/5 upon expression in recombinant hosts, e.g. introducing or moving sites of glycosylation, or introducing membrane anchor sequences (in accordance with PCT WO 89/01041 published Feb. 9, 1989).

In some embodiments, NT-4/5 polypeptide comprises an amino acid sequence encoded by a nucleic acid that hybridizes under stringent conditions to a nucleic acid sequence (e.g., SEQ ID NO:2) encoding mature human NT-4/5.

Variants polynucleotides may also, or alternatively, be substantially homologous to a native gene, or a portion or complement thereof. Such polynucleotide variants are capable of hybridizing under moderately stringent conditions to a naturally occurring DNA sequence encoding a the polypeptide (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Another exemplary stringent condition hybridization in 50% formamide, 5×SSC, 0.1% sodium dodecyl sulfate, 0.1% sodium pyrophosphate, 50 mM sodium phosphate pH 6.8, 2×Denhardt's solution, and 10% dextran sulfate at 42° C., followed by a wash in 0.1×SSC and 0.1% SDS at 42° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

TrkB agonists used in the methods of the invention also include fusion proteins comprising the amino acid sequence of NT-4/5 (e.g., human NT-4/5 shown in Table 1) or a functional peptide fragment thereof. Biologically active NT-4/5 polypeptides can be fused with sequences, such as sequences that enhance immunological reactivity, facilitate the coupling of the polypeptide to a support or a carrier, or facilitate refolding and/or purification (e.g., sequences encoding epitopes such as Myc, HA derived from influenza virus hemagglutinin, His-6, FLAG). These sequences may be fused to NT-4/5 polypeptide at the N-terminal end or at the C-terminal end. In addition, the protein or polynucleotide can be fused to other or polypeptides which increase its function, or specify its localization in the cell, such as a secretion sequence. Methods for producing recombinant fusion proteins described above are known in the art. The recombinant fusion protein can be produced, refolded and isolated by methods well known in the art.

NT-4/5 polypeptides described herein may be modified to increase their half lives in an individual. For example, NT-4/5 polypeptide may be pegylated to reduce systemic clearance with minimal loss of biological activity. The invention also provides compositions (including pharmaceutical compositions) comprising an NT-4/5 polypeptide linked to a PEG molecule. In some embodiments, the PEG molecule is linked to the NT-4/5 polypeptide through a reversible linkage. The half life of a pegylated NT-4/5 polypeptide may be extended by more than about any of 2-fold, 5-fold, 10-fold, 15-fold, 20-fold, and 30-fold of the half life of the non-pegylated NT-4/5 polypeptide.

Polyethylene glycol polymers (PEG) may be linked to various functional groups of the NT-4/5 polypeptide using methods known in the art. See, e.g., Roberts et al., Advanced Drug Delivery Reviews 54:459-476 (2002); Sakane et al. Pharm. Res. 14:1085-91 (1997). PEG may be linked to the following functional groups on the polypeptide: amino groups, carboxyl groups, modified or natural N-termini, amine groups, and thiol groups. In some embodiments, one or more surface amino acid residues are modified with PEG molecules. PEG molecules may be of various sizes (e.g., ranging from about 2 to 40 KDa). PEG molecules linked to NT-4/5 polypeptide may have a molecular weight about any of 2000, 10,000, 15,000, 20,000, 25,000, 30,000, 35,000, 40,000 Da. PEG molecule may be a single or branched chain. To link PEG to NT-4/5 polypeptide, a derivative of the PEG having a functional group at one or both termini may be used. The functional group is chosen based on the type of available reactive group on NT-4/5 polypeptide. Methods of linking derivatives to polypeptides are known in the art. Roberts et al., Advanced Drug Delivery Reviews 54:459-476 (2002). The linkage between the NT-4/5 polypeptide and the PEG may also be such that it can be cleaved or naturally degrades (reversible or degradable linkage) in an individual which may improve the half-life but minimize loss of activity. PEG linking site on NT-4/5 polypeptide may also be created by mutating surface residues to an amino acid residue having a PEG reactive group, such as, a cysteine. For example, the following amino acids of human NT-4/5 (SEQ ID NO:1) may be mutated for PEG attachment: G1, V2, S3, E4, T5, S9, R10, T25, D26, R28, T29, V31, E37, E39, L41, E43, A46, A47, G48, G49, S50, R53, D64, N65, A66, E67, E68, G69, D82, R83, R84, H85, A104, Q105, G106, R107, V108, S125, and T127. These may be applied to the corresponding residues in other species.

Several pegylated NT-4/5 have been generated and are shown in Examples 6 and 7 of US Pat. Appl Pub. No. 2005/0209148 and PCT WO 2005/082401. Serine residue at position 50 of the mature human NT-4/5 may be changed to cysteine to generate NT4-S50C which is then pegylated, wherein the PEG is linked to the cysteine at position 50. One example of an N-terminal specific attachment for PEG is to mutate the residue at position 1 to a serine or threonine, then followed with pegylation, wherein the PEG is linked to the serine at position 1.

NT-4/5 polypeptide can be produced by recombinant means, that is, by expression of nucleic acid encoding the NT-4/5 polypeptide. In recombinant cell culture, and, optionally, purification of the variant polypeptide from the cell culture, for example, by bioassay of the variant's activity or by adsorption on an immunoaffinity column comprising rabbit anti-NT-4/5 polyclonal antibodies (which will bind to at least one immune epitope of the variant which is also present in native NT-4/5). Small peptide fragments, on the order of 40 residues or less, are conveniently made by in vitro methods.

DNA encoding NT-4/5 polypeptide may be cloned into an expression vector for expressing the protein in a host cell. Examples of nucleic acids encoding NT-4/5 polypeptide are described in U.S. Pat. Appl. Pub. No. 2003/0203383. The DNA encoding NT-4/5 polypeptide in its mature form may be linked at its amino terminus to a secretion signal. This secretion signal preferably is the NT-4/5 presequence that normally directs the secretion of NT-4/5 from human cells in vivo. However, suitable secretion signals also include signals from other animal NT-4/5, signals from NGF, NT-2, or NT-3, viral signals, or signals from secreted polypeptides of the same or related species. Any host cell (such as E. coli) may be used for expressing the protein or polypeptide.

NT-4/5 polypeptide expressed may be purified. NT-4/5 polypeptide may be recovered from the culture medium as a secreted protein, although it also may be recovered from host cell lysates when directly expressed without a secretory signal. Protein purification methods known in the art may be used. Methods of producing NT-4/5 polypeptide and purifying the expressed NT-4/5 polypeptide are described in U.S. Pat. Appl. Pub. No. 2003/0203383, and U.S. Pat. No. 6,184,360. NT-4/5 polypeptide can be expressed in E. coli and refolded according to methods known in the art. Mature human NT-4/5 may also be obtained commercially (for example, from R&D Systems, Sigma and Upstate).

Anti-trkB Agonist Polypeptides and Antibodies

The trkB agonist used in the methods of the invention also includes anti-trkB agonist polypeptides, including anti-trkB agonist antibodies. An anti-trkB agonist polypeptide (e.g., an antibody) should exhibit any one or more of the following characteristics: (a) bind to trkB receptor; (b) bind to trkB receptor and activate trkB biological activity(ies) and/or one or more downstream pathways mediated by trkB signaling function(s); (c) bind to trkB receptor and increase body weight and/or food intake in a primate when administered peripherally; (d) bind to trkB receptor and treat, prevent, reverse, or ameliorate one or more symptoms of cachexia in a primate when administered peripherally; (e) bind to trkB receptor and treat, prevent, reverse, or ameliorate one or more symptoms of anorexia nervosa in a primate when administered peripherally; (f) bind to trkB receptor and treat, prevent, reverse, or ameliorate one or more symptoms of opioid-induced emesis in a mammal when administered peripherally; (g) promote trkB receptor dimerization and activation; and (h) increase trkB receptor-dependent neuronal survival and/or neurite outgrowth.

In some embodiments, the anti-trkB agonist polypeptide (e.g., antibody) is multivalent and binds to the extracellular domain of a trkB receptor. It has been shown that immunoglobulins that are able to bind and cross-link or dimerize the trk family of neurotrophin-receptors activate these receptors and produce consequences in neurons that are similar to exposure to a neurotrophin. See, U.S. Pat. No. 6,656,465; and PCT WO 01/98361.

The trkB agonist antibodies can encompass monoclonal antibodies, polyclonal antibodies, antibody fragments (e.g., Fab, Fab′, F(ab′)₂, Fv, Fc, etc.), chimeric antibodies, single chain (ScFv), mutants thereof, fusion proteins comprising an antibody portion, and any other modified configuration of the immunoglobulin molecule that comprises an antigen recognition site of the required specificity. The antibodies may be murine, rat, human, or any other origin (including humanized antibodies).

In some embodiments, the polypeptide (including the antibody) binds trkB and does not significantly cross-react (bind) with other neurotrophin receptors (such as the related neurotrophin receptors, trkA and/or trkC). The agonist anti-trkB polypeptide may bind human trkB. The agonist anti-trkB polypeptide may also bind human and rodent trkB. In some embodiments, the agonist anti-trkB polypeptide may bind human and rat trkB. In some embodiments, the anti-trkB polypeptide may bind human and mouse trkB. In one embodiment, the polypeptide recognizes one or more epitopes on human trkB extracellular domain. In another embodiment, the antibody is a mouse or rat antibody that recognizes one or more epitopes on human trkB extracellular domain. In some embodiments, the polypeptide binds human trkB and does not significantly bind trkB from another mammalian species (in some embodiments, vertebrate species). In some embodiments, the polypeptide binds human trkB as well as one or more trkB from another mammalian species (in some embodiments, vertebrate species). In another embodiment, the polypeptide recognizes one or more epitopes on a trkB selected from one or more of: primate, canine, feline, equine, and bovine.

In some embodiments, the anti-trkB agonist antibody has an EC₅₀ (half of the maximal effective concentration) of less than about any of 0.01 nM, 0.1 nM, 0.5 nM, 1 nM, 5 nM, 10 nM, 50 nM, or 100 nM in TrkB receptor (e.g., human trkB) activation in vitro (e.g., assays described in Example 6, and in US 2005/0209148 and PCT WO 2005/082401).

The binding affinity of anti-trkB agonist polypeptide (e.g., antibody) to trkB may be any of about 500 nM, about 400 nM, about 300 nM, about 200 nM, about 100 nM, about 50 nM, about 10 nM, about 1 nM, about 500 pM, about 100 pM, or about 50 pM to any of about 2 pM, about 5 pM, about 10 pM, about 15 pM, about 20 pM, or about 40 pM. In some embodiments, the binding affinity is any of about 100 nM, about 50 nM, about 10 nM, about 1 nM, about 500 pM, about 100 pM, or about 50 pM, or less than about 50 pM. In some embodiments, the binding affinity is less than any of about 100 nM, about 50 nM, about 10 nM, about 1 nM, about 500 pM, about 100 pM, or about 50 pM. In still other embodiments, the binding affinity is about 2 pM, about 5 pM, about 10 pM, about 15 pM, about 20 pM, about 40 pM, or greater than about 40 pM. As is well known in the art, binding affinity can be expressed as K_{D}, or dissociation constant, and an increased binding affinity corresponds to a decreased K_{D}.

One way of determining binding affinity of antibodies to trkB is by measuring binding affinity of monofunctional Fab fragments of the antibody. To obtain monofunctional Fab fragments, an antibody (for example, IgG) can be cleaved with papain or expressed recombinantly. The affinity of an anti-trkB Fab fragment of an antibody can be determined by surface plasmon resonance (BIAcore3000™ surface plasmon resonance (SPR) system, BIAcore, INC, Piscataway N.J.). CM5 chips can be activated with N-ethyl-N′-(3-dimethylaminopropyl)-carbodiinide hydrochloride (EDC) and N-hydroxysuccinimide (NHS) according to the supplier's instructions. Human trkB-Fc fusion protein (“htrkB”) (or any other trkB, such as rat trkB) can be diluted into 10 mM sodium acetate pH 5.0 and injected over the activated chip at a concentration of 0.0005 mg/mL. Using variable flow time across the individual chip channels, two ranges of antigen density can be achieved: 200-400 response units (RU) for detailed kinetic studies and 500-1000 RU for screening assays. The chip can be blocked with ethanolamine. Regeneration studies have shown that a mixture of Pierce elution buffer (Product No. 21004, Pierce Biotechnology, Rockford, Ill.) and 4 M NaCl (2:1) effectively removes the bound Fab while keeping the activity of htrkB on the chip for over 200 injections. HBS-EP buffer (0.01M HEPES, pH 7.4, 0.15 NaCl, 3 mM EDTA, 0.005% Surfactant P29) is used as running buffer for the BIAcore assays. Serial dilutions (0.1-10× estimated K_{D}) of purified Fab samples are injected for 1 min at 100 μL/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) (generally measured at 25° C.) are obtained simultaneously by fitting the data to a 1:1 Langmuir binding model (Karlsson, R. Roos, H. Fagerstam, L. Petersson, B. (1994). Methods Enzymology 6:99-110) using the BIAevaluation program. Equilibrium dissociation constant (K_{D}) values are calculated as k_{off}/kₒₙ.

In some embodiments, the anti-trkB agonist polypeptide (including antibody) has impaired effector function. As used herein, an antibody or a polypeptide having an “impaired effector function” (used interchangeably with the term “immunologically inert”) refers to antibodies or polypeptides that do not have any effector function or have reduced activity or activities of effector function (compared to antibody or polypeptide having an unmodified or a naturally occurring constant region), e.g., having no activity or reduced activity in any one or more of the following: a) triggering complement mediated lysis; b) stimulating antibody-dependent cell mediated cytotoxicity (ADCC); and c) activating microglia. The effector function activity may be reduced by about any of 10%, 20%, 30%, 40%, 50%, 60%, 70%, 80%, 90%, 95%, 99%, and 100%. In some embodiments, the antibody binds to trkB receptor without triggering significant complement dependent lysis, or cell mediated destruction of the target. For example, the Fc receptor binding site on the constant region may be modified or mutated to remove or reduce binding affinity to certain Fc receptors, such as FcγRI, FcγRII, FcγRIII, and/or FcγRIV. For simplicity, reference will be made to antibodies with the understanding that embodiments also apply to polypeptides. EU numbering system (Kabat et al., Sequences of Proteins of Immunological Interest; 5th ed. Public Health Service, National Institutes of Healthy, Bethesda, Md., 1991) is used to indicate which amino acid residue(s) of the constant region (e.g., of an IgG antibody) are altered or mutated. The numbering may be used for a specific type of antibody (e.g., IgG1) or a species (e.g., human) with the understanding that similar changes can be made across types of antibodies and species.

In some embodiments, the polypeptides (including antibodies) that specifically bind to a trkB receptor comprise a heavy chain constant region having impaired effector function. The heavy chain constant region may have naturally occurring sequence or is a variant. In some embodiments, the amino acid sequence of a naturally occurring heavy chain constant region is mutated, e.g., by amino acid substitution, insertion and/or deletion, whereby the effector function of the constant region is impaired. In some embodiments, the N-glycosylation of the Fc region of a heavy chain constant region may also be changed, e.g., may be removed completely or partially, whereby the effector function of the constant region is impaired.

In some embodiments, the effector function is impaired by removing N-glycosylation of the Fc region (e.g., in the CH 2 domain of IgG). In some embodiments, N-glycosylation of the Fc region is removed by mutating the glycosylated amino acid residue or flanking residues that are part of the glycosylation recognition sequence in the constant region. The tripeptide sequences asparagine-X-serine (N-X-S), asparagine-X-threonine (N-X-T) and asparagine-X-cysteine (N-X-C), where X is any amino acid except proline, are the recognition sequences for enzymatic attachment of the carbohydrate moiety to the asparagine side chain for N-glycosylation. Mutating any of the amino acid in the tripeptide sequences in the constant region yields an aglycosylated IgG. For example, N-glycosylation site N297 of human IgG1 and IgG3 may be mutated to A, D, Q, K, or H. See, Tao et al., J. Immunology 143: 2595-2601 (1989); and Jefferis et al., Immunological Reviews 163:59-76 (1998). It has been reported that human IgG1 and IgG3 with substitution of Asn-297 with Gln, His, or Lys do not bind to the human FcγRI and do not activate complement with C1q binding ability completely lost for IgG1 and dramatically decreased for IgG3. In some embodiments, the amino acid N in the tripeptide sequences is mutated to any one of amino acid A, C, D, E, F, G, H, I, K, L, M, P, Q, R, S, T, V, W, Y. In some embodiments, the amino acid N in the tripeptide sequences is mutated to a conservative substitution. In some embodiments, the amino acid X in the tripeptide sequences is mutated to proline. In some embodiments, the amino acid S in the tripeptide sequences is mutated to A, D, E, F, G, H, I, K, L, M, N, P, Q, R, V, W, Y. In some embodiments, the amino acid T in the tripeptide sequences is mutated to A, D, E, F, G, H, I, K, L, M, N, P, Q, R, V, W, Y. In some embodiments, the amino acid C in the tripeptide sequences is mutated to A, D, E, F, G, H, I, K, L, M, N, P, Q, R, V, W, Y. In some embodiments, the amino acid following the tripeptide is mutated to P. In some embodiments, the N-glycosylation in the constant region is removed enzymatically (such as N-glycosidase F, endoglycosidase F1, endoglycosidase F2, endoglycosidase F3, and englycosidase H). Removing N-glycosylation may also be achieved by producing the antibody in a cell line having deficiency for N-glycosylation. Wright et al., J. Immunol. 160(7):3393-402 (1998).

In some embodiments, amino acid residue interacting with oligosaccharide attached to the N-glycosylation site of the constant region is mutated to reduce binding affinity to FcγRI. For example, F241, V264, D265 of human IgG3 may be mutated. See, Lund et al., J. Immunology 157:4963-4969 (1996).

In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000). Antibodies described in PCT WO 99/58572 and Armour et al. comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain has a reduced affinity for FcγRI, FcγRIIa, and FcγRIII. In some embodiments, the effector domain is capable of specifically binding FcRn and/or FcγRIIb. These are typically based on chimeric domains derived from two or more human immunoglobulin heavy chain C_{H}2 domains. Antibodies modified in this manner are particularly suitable for use in chronic antibody therapy, to avoid inflammatory and other adverse reactions to conventional antibody therapy. In some embodiments, the heavy chain constant region of the antibody is a human heavy chain IgG1 with any of the following mutations: 1) A327A330P331 to G327S330S331; 2) E233L234L235G236 to P233V234A235 with G236 deleted; 3) E233L234L235 to P233V234A235; 4) E233L234L235G236A327A330P331 to P233V234A235G327S330S331 with G236 deleted; 5) E233L234L235A327A330P331 to P233V234A235G327S330S331; and 6) N297 to A297 or any other amino acid except N. In some embodiments, the heavy chain constant region of the antibody is a human heavy chain IgG2 with the following mutations: A330P331 to S330S331. In some embodiments, the heavy chain constant region of the antibody is a human heavy chain IgG4 with any of the following mutations: E233F234L235G236 to P233V234A235 with G236 deleted; E233F234L235 to P233V234A235; and S228L235 to P228E235.

The constant region may also be modified to impair complement activation. For example, complement activation of IgG antibodies following binding of the C1 component of complement may be reduced by mutating amino acid residues in the constant region in a C1 binding motif (e.g., C1q binding motif). It has been reported that Ala mutation for each of D270, K322, P329, P331 of human IgG1 significantly reduced the ability of the antibody to bind to C1q and activating complement. For murine IgG2b, C1q binding motif constitutes residues E318, K320, and K322. Idusogie et al., J. Immunology 164:4178-4184 (2000); Duncan et al., Nature 322: 738-740 (1988).

CIq binding motif E318, K320, and K322 identified for murine IgG2b is believed to be common for other antibody isotypes. Duncan et al., Nature 322: 738-740 (1988). C1q binding activity for IgG2b can be abolished by replacing any one of the three specified residues with a residue having an inappropriate functionality on its side chain. It is not necessary to replace the ionic residues only with Ala to abolish Clq binding. It is also possible to use other alkyl-substituted non-ionic residues, such as Gly, Ile, Leu, or Val, or such aromatic non-polar residues as Phe, Tyr, Trp and Pro in place of any one of the three residues in order to abolish CIq binding. In addition, it is also be possible to use such polar non-ionic residues as Ser, Thr, Cys, and Met in place of residues 320 and 322, but not 318, in order to abolish CIq binding activity.

The invention also provides antibodies having impaired effector function wherein the antibody has a modified hinge region. Binding affinity of human IgG for its Fc receptors can be modulated by modifying the hinge region. Canfield et al., J. Exp. Med. 173:1483-1491 (1991); Hezareh et al., J. Virol. 75:12161-12168 (2001); Redpath et al., Human Immunology 59:720-727 (1998). Specific amino acid residues may be mutated or deleted. The modified hinge region may comprise a complete hinge region derived from an antibody of different antibody class or subclass from that of the CH1 domain. For example, the constant domain (CH1) of a class IgG antibody can be attached to a hinge region of a class IgG4 antibody. Alternatively, the new hinge region may comprise part of a natural hinge or a repeating unit in which each unit in the repeat is derived from a natural hinge region. In some embodiments, the natural hinge region is altered by converting one or more cysteine residues into a neutral residue, such as alanine, or by converting suitably placed residues into cysteine residues. U.S. Pat. No. 5,677,425. Such alterations are carried out using art recognized protein chemistry and, preferably, genetic engineering techniques and as described herein.

Polypeptides that specifically bind to a trkB receptor and fused to a heavy chain constant region having impaired effector function may also be used for the methods described herein. An example of such fusion polypeptides is an immunoadhesin. See, e.g., U.S. Pat. No. 6,153,189.

Other methods to make antibodies having impaired effector function known in the art may also be used.

Antibodies and polypeptides with modified constant regions can be tested in one or more assays to evaluate level of effector function reduction in biological activity compared to the starting antibody. For example, the ability of the antibody or polypeptide with an altered Fc region to bind complement or Fc receptors (for example, Fc receptors on microglia), or altered hinge region can be assessed using the assays disclosed herein as well as any art recognized assay. PCT WO 99/58572; Armour et al., Molecular Immunology 40: 585-593 (2003); Reddy et al., J. Immunology 164:1925-1933 (2000); Song et al., Infection and Immunity 70:5177-5184 (2002).

The anti-trkB agonist antibodies may be made by using immunogens which express one or more extracellular domains of trkB. One example of an immunogen is cells with high expression of trkB, which can be obtained as described herein. Another example of an immunogen that can be used is a soluble protein (such as a trkB immunoadhesin) which contains the extracellular domain or a portion of the extracellular domain of trkB receptor.

The route and schedule of immunization of the host animal are generally in keeping with established and conventional techniques for antibody stimulation and production, as further described herein. General techniques for production of human and mouse antibodies are known in the art and are described herein.

It is contemplated that any mammalian subject including humans or antibody producing cells therefrom can be manipulated to serve as the basis for production of mammalian, including human, hybridoma cell lines. Typically, the host animal is inoculated intraperitoneally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W. et al., (1982) In Vitro, 18:377-381. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-trkB monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for trkB, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a human or other species of trkB receptor, or a fragment of the human or other species of trkB receptor, or a human or other species of trkB receptor or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaradehyde, succinic anhydride, SOCl2, or R1N═C═NR, where R and R1 are different alkyl groups can yield a population of antibodies (e.g., monoclonal antibodies). Another example of an immunogen is cells with high expression of trkB, which can be obtained from recombinant means, or by isolating or enriching cells from a natural source that express a high level of trkB. These cells may be of human or other animal origin, and may be used as an immunogen as directly isolated, or may be processed in such that immunogenicity is increased, or trkB expression (of a fragment of trkB) is increased or enriched. Such processing includes, but is not limited to, treatment of the cells or fragments thereof with agents designed to increase their stability or immunogenicity, such as, e.g., formaldehyde, glutaraldehyde, ethanol, acetone, and/or various acids. Further, either before or after such treatment the cells may be processed in order to enrich for the desired immunogen, in this case trkB or fragment thereof. These processing steps can include membrane fractionation techniques, which are well known in the art.

If desired, the anti-trkB antibody (monoclonal or polyclonal) of interest may be sequenced and the polynucleotide sequence may then be cloned into a vector for expression or propagation. The sequence encoding the antibody of interest may be maintained in a vector in a host cell and the host cell can then be expanded and frozen for future use. As an alternative, the polynucleotide sequence may be used for genetic manipulation to “humanize” the antibody or to improve the affinity, or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to trkB receptor and greater efficacy in activating trkB receptor. It will be apparent to one of skill in the art that one or more polynucleotide changes can be made to the anti-trkB antibody and still maintain its binding ability to trkB extracellular domain or epitopes of trkB.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; and 6,548,640. For example, the constant region may be engineered to more resemble human constant regions to avoid immune response if the antibody is used in clinical trials and treatments in humans. See, for example, U.S. Pat. Nos. 5,997,867 and 5,866,692.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J. Immunol. 138:4534-4538 (1987), and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988), Verhoeyen et al. Science 239:1534-1536 (1988), and Jones et al. Nature 321:522-525 (1986). Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules which limits the duration and effectiveness of therapeutic applications of those moieties in human recipients. The antibody constant region can be engineered such that it is immunologically inert, e.g., does not trigger a complement mediated lysis or does not stimulate antibody-dependent cell mediated cytotoxicity (ADCC). In other embodiments, the constant region is modified as described in Eur. J. Immunol. (1999) 29:2613-2624; PCT Application No. PCT/GB99/01441; and/or UK Patent Application No. 9809951.8.

See, e.g. PCT/GB99/01441; UK patent Application No. 9809951.8. Other methods of humanizing antibodies that may also be utilized are disclosed by Daugherty et al., Nucl. Acids Res. 19:2471-2476 (1991) and in U.S. Pat. Nos. 6,180,377; 6,054,297; 5,997,867; 5,866,692; 6,210,671; 6,350,861; and PCT Publication No. WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743 and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S, and Chiswell, David J., Current Opinion in Structural Biology 3, 564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines and bovines.

The antibody may be a bispecific antibody, a monoclonal antibody that has binding specificities for at least two different antigens, can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539).

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690, published Mar. 3, 1994.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; and EP 03089). Heteroconjugate antibodies may be made using any convenient cross-linking methods. Suitable cross-linking agents and techniques are well known in the art, and are described in U.S. Pat. No. 4,676,980.

Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants (e.g., tobacco), transgenic milk, or in other organisms. Methods for expressing antibodies recombinantly in plants or milk have been disclosed. See, for example, Peeters et al. (2001) Vaccine 19:2756; Lonberg, N. and D. Huszar (1995) Int. Rev. Immunol 13:65; and Pollock et al. (1999) J Immunol Methods 231:147. Methods for making derivatives of antibodies, e.g., humanized, single chain, etc. are known in the art.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Single chain Fv fragments may also be produced, such as described in Iliades et al., 1997, FEBS Letters, 409:437-441. Coupling of such single chain fragments using various linkers is described in Kortt et al., 1997, Protein Engineering, 10:423-433. A variety of techniques for the recombinant production and manipulation of antibodies are well known in the art.

Antibodies may be modified as described in PCT Publication No. WO 99/58572, published Nov. 18, 1999. These antibodies comprise, in addition to a binding domain directed at the target molecule, an effector domain having an amino acid sequence substantially homologous to all or part of a constant domain of a human immunoglobulin heavy chain. These antibodies are capable of binding the target molecule without triggering significant complement dependent lysis, or cell-mediated destruction of the target. Preferably, the effector domain is capable of specifically binding FcRn and/or FcγRIIb. These are typically based on chimeric domains derived from two or more human immunoglobulin heavy chain C_{H}2 domains. Antibodies modified in this manner are preferred for use in chronic antibody therapy, to avoid inflammatory and other adverse reactions to conventional antibody therapy.

The antibodies made either by immunization of a host animal or recombinantly should exhibit any one or more of the trkB agonist activities described herein.

Immunoassays and flow cytometry sorting techniques such as fluorescence activated cell sorting (FACS) can also be employed to isolate antibodies that are specific for trkB.

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

DNA encoding agonist anti-trkB antibodies may be sequenced, as is known in the art. Generally, the monoclonal antibody is readily isolated and sequenced using conventional procedures (e.g., by using oligonucleotide probes that are capable of binding specifically to genes encoding the heavy and light chains of the monoclonal antibodies). The hybridoma cells serve as a preferred source of such cDNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, Morrison et al., Proc. Nat. Acad. Sci. 81: 6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-trkB monoclonal antibody herein. The DNA encoding the agonist anti-trkB antibody (such as an antigen binding fragment thereof) may also be used for delivery and expression of agonist anti-trkB antibody in a desired cell, as described here. DNA delivery techniques are further described herein.

Anti-trkB antibodies may be characterized using methods well-known in the art. For example, one method is to identify the epitope to which it binds, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an anti-trkB antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an anti-trkB antibody. In another example, the epitope to which the anti-trkB antibody binds can be determined in a systematic screening by using overlapping peptides derived from the trkB extracellular sequence and determining binding by the anti-trkB antibody. According to the gene fragment expression assays, the open reading frame encoding trkB is fragmented either randomly or by specific genetic constructions and the reactivity of the expressed fragments of trkB with the antibody to be tested is determined. The gene fragments may, for example, be produced by PCR and then transcribed and translated into protein in vitro, in the presence of radioactive amino acids. The binding of the antibody to the radioactively labeled trkB fragments is then determined by immunoprecipitation and gel electrophoresis. Certain epitopes can also be identified by using large libraries of random peptide sequences displayed on the surface of phage particles (phage libraries).

Yet another method which can be used to characterize an anti-trkB antibody is to use competition assays with other antibodies known to bind to the same antigen, i.e., trkB extracellular domain to determine if the anti-trkB antibody binds to the same epitope as other antibodies. Competition assays are well known to those of skill in the art. Examples of antibodies useful in competition assays include the following: antibodies 6.1.2, 6.4.1, 2345, 2349, 2.5.1, 2344, 2248, 2250, 2253, and 2256. See PCT Publication No. WO 01/98361

Epitope mapping can also be performed using domain swap mutants as described in PCT Publication No. WO 01/98361. Generally, this approach is useful for anti-trkB antibodies that do not significantly cross-react with trkA or trkC. Domain-swap mutants of trkB can be made by replacing extracellular domains of trkB with the corresponding domains from trkC or trkA. The binding of each agonist anti-trkB antibody to various domain-swap mutants can be evaluated and compared to its binding to wild type (native) trkB using ELISA or other method known in the art. In another approach, alanine scanning can be performed. Individual residues of the antigen, the trkB receptor, are systematically mutated to another amino acid (usually alanine) and the effect of the changes is assessed by testing the ability of the modified trkB to bind to antibody using ELISA or other methods known in the art.

BDNF Polypeptides

The trkB agonist used in the methods of the invention includes BDNF polypeptides. As used herein, “BDNF polypeptide” includes naturally-occurring mature protein (interchangeably termed “BDNF”) such as mature human BDNF shown in U.S. Pat. No. 5,180,820 and naturally occurring amino acid sequence variants of BDNF; amino acid sequence variants of BDNF; peptide fragments of mature BDNF (such as human) and said amino acid sequence variants; and modified forms of mature BDNF and said amino acid sequence variants and peptide fragments wherein the polypeptide or peptide has been covalently modified by substitution with a moiety other than a naturally occurring amino acid, as long as the amino acid sequence variant, peptide fragment, and the modified form thereof show one or more biological activities of a trkB agonist and/or of naturally occurring mature BDNF protein. TrkB agonists also include fusion proteins and conjugates comprising any of the BDNF polypeptide embodiments described herein, e.g., an BDNF polypeptide conjugated or fused to a half life extending moiety, such as a PEG or a peptide. The amino acid sequence variants, peptide fragments (including fragments of variants), or modified forms thereof under consideration do not include NGF, NT-4/5, or NT-3 of any animal species. BDNF polypeptides include any one or more embodiments described herein. For example, BDNF polypeptide comprises a naturally occurring sequence with one or more amino acid insertion, deletion, or substitution.

In some embodiments, the BDNF polypeptide is a mammalian BDNF polypeptide which may be a naturally occurring mammalian BDNF, or BDNF polypeptide derived from a naturally occurring mammalian BDNF and having a sequence that does not match any part of a naturally occurring non-mammalian BDNF. In some embodiments, the BDNF polypeptide is a human BDNF polypeptide which may be a naturally occurring human BDNF, or BDNF polypeptide derived from a naturally occurring human BDNF and having a sequence that does not match any part of a naturally occurring non-human BDNF.

BDNF polypeptides, including variants, peptide fragments, modified forms of BDNF polypeptides (including naturally occurring BDNF), fusion protein and conjugate of the invention are characterized by any (one or more) of the following characteristics: (a) bind to trkB receptor; (b) bind to trkB receptor and activate trkB biological activity(ies) and/or one or more downstream pathways mediated by trkB signaling function(s); (c) bind to trkB receptor and increase body weight and/or food intake in a primate when administered peripherally; (d) bind to trkB receptor and treat, prevent, reverse, or ameliorate one or more symptoms of cachexia in a primate when administered peripherally; (e) bind to trkB receptor and treat, prevent, reverse, or ameliorate one or more symptoms of anorexia nervosa in a primate when administered peripherally; (f) bind to trkB receptor and treat, prevent, reverse, or ameliorate one or more symptoms of opioid-induced emesis in a mammal when administered peripherally; (g) promote trkB receptor dimerization and activation; and (h) increase trkB receptor-dependent neuronal survival and/or neurite outgrowth. Thus all BDNF polypeptides (including variants, fragments, and modified forms) are functional as described above.

Biological activity of variants may be tested in vitro and in vivo using methods known in the art and methods described herein. BDNF polypeptides may have an enhanced activity or reduced activity as compared to a naturally occurring BDNF protein. In some embodiments, functionally equivalent variants have at least about any of 50%, 60%, 70%, 75%, 80%, 85%, 90%, or 95% of activity as compared to the native BDNF protein from which the BDNF polypeptide is derived with respect to one or more of the biological assays described above (or known in the art). In some embodiments, functionally equivalent variants have an EC₅₀ (half of the maximal effective concentration) of less than about any of 0.01 nM, 0.1 nM, 1 nM, 10 nM, or 100 nM in TrkB receptor activation in vitro (e.g., assays described in Example 6, and in US 2005/0209148 and PCT WO 2005/082401).

Amino acid sequence variants of BDNF include polypeptides having an amino acid sequence which differs from naturally occurring BDNF by virtue of the insertion, deletion, and/or substitution of one or more amino acid residues within the sequence of naturally occurring BDNF (for example, mature human BDNF). Amino acid sequence variants generally will be at least about 65%, 70%, 75%, 80%, 85%, 90%, 95%, 96%, 97%, 98%, or 99% identical to any naturally occurring BDNF (such as mature human BDNF). In some embodiments, the variant is at least about 70% identical to the amino acid sequence of mature human BDNF. In some embodiments, the variant is at least about 85% identical to the amino acid sequence of mature human BDNF. In some embodiments, the variant is at least about 90% identical to the amino acid sequence of mature human BDNF. In some embodiments, the variant is at least about 95% identical to the amino acid sequence of mature human BDNF.

Obviously, such variations which, for example, convert BDNF into NGF, BDNF, or NT-3 are not included within the scope of this invention. Thus, while the site for introducing an amino acid sequence variation is predetermined, the nature of the mutation per se need not be predetermined. For example, to optimize the performance of a mutation at a given site, ala scanning or random mutagenesis is conducted at the target codon or region and the expressed BDNF variants are screened for the optimal desired activity.

Amino acid sequence deletions generally range from about 1 to 30 residues, more preferably, about 1 to 10 residues, and typically are contiguous. Deletions may be introduced into regions of low homology among BDNF, NGF, NT-3, and NT-4/5 to modify the activity of BDNF. Deletions from BDNF in areas of substantial homology with NT-4/5, NT-3, and NGF may be more likely to modify the biological activity of BDNF more significantly. The number of consecutive deletions may be selected so as to preserve the tertiary structure of BDNF in the affected domain, e.g., beta-pleated sheet or alpha helix.

Amino acid sequence insertions include amino- and/or carboxyl-terminal fusions ranging in length from one residue to polypeptides containing a thousand or more residues, as well as intrasequence insertions of single or multiple amino acid residues. Intrasequence insertions (i.e., insertions within the mature BDNF sequence) may range generally from about 1 to 10 residues, more preferably, 1 to 5, most preferably 1 to 3. An example of a terminal insertion includes fusion of a heterologous N-terminal signal sequence to the N-terminus of the BDNF molecule to facilitate the secretion of mature BDNF from recombinant host. Such signals generally will be homologous to the intended host cell and include STII or Ipp for E. coli, alpha factor for yeast, and viral signals such as herpes gD for mammalian cells. Other insertions include the fusion of a polypeptide to the N- or C-termini of BDNF.

Another group of variants includes those in which at least one amino acid residue in BDNF, and preferably only one, has been removed and a different residue inserted in its place. An example is the replacement of arginine and lysine by other amino acids to render the BDNF resistant to proteolysis by serine proteases, thereby creating a variant of BDNF that is more stable. The sites of greatest interest for substitutional mutagenesis include sites where the amino acids found in BDNF, NGF, NT-3, and NT-4/5 are substantially different in terms of side chain bulk, charge or hydrophobicity, but where there also is a high degree of homology at the selected site within various animal analogues of NGF, NT-3, and NT-4/5 (e.g. among all the animal NGFs, all the animal NT-3, and all the BDNFs). This analysis will highlight residues that may be involved in the differentiation of activity of the trophic factors, and therefore, variants at these sites may affect such activities. Other sites of interest are those in which the residues are identical among all animal species BDNF, NGF, NT-3, and NT-4/5, this degree of conformation suggesting importance in achieving biological activity common to all four factors.

For example, substitution of one or more amino acids includes conservative substitutions. Methods of making conservative substitutions are known in the art. For example, ala (A) may be substituted by val, leu, ile, preferably by val; arg (R) may be substituted by lys, gin, asn, preferably by lys; asn (N) may be substituted by gin, his, lys, arg, preferably by gin; asp (D) may be substituted by glu; cys (C) may be substituted by ser; gin (O) may be substituted by asn; glu (E) may be substituted by asp; gly (G) may be substituted by pro; his (H) may be substituted by asn, gin, lys, arg; preferably by arg; ile (I) may be substituted by leu, val, met, ala, phe, norleucine, preferably by leu; leu (L) may be substituted by norleucine, ile, val, met; ala; phe, preferably by ile; lys (K) may be substituted by arg; gin, asn, preferably by arg; met (M) may be substituted by leu; phe; ile, preferably by leu; phe (F) may be substituted by leu, val, ile, ala, preferably by leu; pro (P) may be substituted by gly; ser (S) may be substituted by thr; thr (T) may be substituted by ser; trp (W) may be substituted by tyr; tyr (Y) may be substituted by trp, phe, thr, ser, preferably by phe; val (V) may be substituted by ile; leu; met; phe, ala; norleucine, preferably by leu.

Substantial modifications in function may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side chain properties (some of these may fall into several functional groups):
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another.

Amino acid sequence variants of BDNF may be naturally occurring or may be prepared synthetically, such as by introducing appropriate nucleotide changes into a previously isolated BDNF DNA, or by in vitro synthesis of the desired variant polypeptide. As indicated above, such variants may comprise deletions from, or insertions or substitutions of, one or more amino acid residues within the amino acid sequence of mature BDNF (e.g., sequence shown in Table 1). Any combination of deletion, insertion, and substitution is made to arrive at an amino acid sequence variant of BDNF, provided that the resulting variant polypeptide possesses a desired characteristic. The amino acid changes also may result in further modifications of BDNF upon expression in recombinant hosts, e.g. introducing or moving sites of glycosylation, or introducing membrane anchor sequences (in accordance with PCT WO 89/01041 published Feb. 9, 1989).

In some embodiments, BDNF polypeptide comprises an amino acid sequence encoded by a nucleic acid that hybridizes under stringent conditions to a nucleic acid sequence encoding mature human BDNF.

Variants polynucleotides may also, or alternatively, be substantially homologous to a native gene, or a portion or complement thereof. Such polynucleotide variants are capable of hybridizing under moderately stringent conditions to a naturally occurring DNA sequence encoding a the polypeptide (or a complementary sequence).

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

TrkB agonists used in the methods of the invention also include fusion proteins comprising the amino acid sequence of BDNF (e.g., human BDNF) or a functional peptide fragment thereof. Biologically active BDNF polypeptides can be fused with sequences, such as sequences that enhance immunological reactivity, facilitate the coupling of the polypeptide to a support or a carrier, or facilitate refolding and/or purification (e.g., sequences encoding epitopes such as Myc, HA derived from influenza virus hemagglutinin, His-6, FLAG). These sequences may be fused to BDNF polypeptide at the N-terminal end or at the C-terminal end. In addition, the protein or polynucleotide can be fused to other or polypeptides which increase its function, or specify its localization in the cell, such as a secretion sequence. Methods for producing recombinant fusion proteins described above are known in the art. The recombinant fusion protein can be produced, refolded and isolated by methods well known in the art.

BDNF polypeptides described herein may be modified to increase their half lives in an individual. For example, BDNF polypeptide may be pegylated to reduce systemic clearance with minimal loss of biological activity. The invention also provides compositions (including pharmaceutical compositions) comprising an BDNF polypeptide linked to a PEG molecule. In some embodiments, the PEG molecule is linked to the BDNF polypeptide through a reversible linkage. The half life of a pegylated BDNF polypeptide may be extended by more than about any of 2-fold, 5-fold, 10-fold, 15-fold, 20-fold, and 30-fold of the half life of the non-pegylated BDNF polypeptide.

Polyethylene glycol polymers (PEG) may be linked to various functional groups of the BDNF polypeptide using methods known in the art. See, e.g., Roberts et al., Advanced Drug Delivery Reviews 54:459-476 (2002); Sakane et al. Pharm. Res. 14:1085-91 (1997). PEG may be linked to the following functional groups on the polypeptide: amino groups, carboxyl groups, modified or natural N-termini, amine groups, and thiol groups. In some embodiments, one or more surface amino acid residues are modified with PEG molecules. PEG molecules may be of various sizes (e.g., ranging from about 2 to 40 KDa). PEG molecules linked to BDNF polypeptide may have a molecular weight about any of 2000, 10,000, 15,000, 20,000, 25,000, 30,000, 35,000, 40,000 Da. PEG molecule may be a single or branched chain. To link PEG to BDNF polypeptide, a derivative of the PEG having a functional group at one or both termini may be used. The functional group is chosen based on the type of available reactive group on BDNF polypeptide. Methods of linking derivatives to polypeptides are known in the art. Roberts et al., Advanced Drug Delivery Reviews 54:459-476 (2002). The linkage between the BDNF polypeptide and the PEG may also be such that it can be cleaved or naturally degrades (reversible or degradable linkage) in an individual which may improve the half-life but minimize loss of activity. PEG linking site on BDNF polypeptide may also be created by mutating surface residues to an amino acid residue having a PEG reactive group, such as, a cysteine.

BDNF polypeptide can be produced by recombinant means, that is, by expression of nucleic acid encoding the BDNF polypeptide. In recombinant cell culture, and, optionally, purification of the variant polypeptide from the cell culture, for example, by bioassay of the variant's activity or by adsorption on an immunoaffinity column comprising rabbit anti-BDNF polyclonal antibodies (which will bind to at least one immune epitope of the variant which is also present in native BDNF). Small peptide fragments, on the order of 40 residues or less, are conveniently made by in vitro methods.

DNA encoding BDNF polypeptide may be cloned into an expression vector for expressing the protein in a host cell. Examples of nucleic acids encoding BDNF polypeptide are described in U.S. Pat. Appl. Pub. No. 2003/0203383. The DNA encoding BDNF polypeptide in its mature form may be linked at its amino terminus to a secretion signal. This secretion signal preferably is the BDNF presequence that normally directs the secretion of BDNF from human cells in vivo. However, suitable secretion signals also include signals from other animal BDNF, signals from NGF, NT-2, or NT-3, viral signals, or signals from secreted polypeptides of the same or related species. Any host cell (such as E. coli) may be used for expressing the protein or polypeptide.

BDNF polypeptide expressed may be purified. BDNF polypeptide may be recovered from the culture medium as a secreted protein, although it also may be recovered from host cell lysates when directly expressed without a secretory signal. Protein purification methods known in the art may be used. Methods of producing BDNF polypeptide and purifying the expressed BDNF polypeptide are known in the art. BDNF polypeptide can be expressed in E. coli and refolded according to methods known in the art. Mature human BDNF may also be obtained commercially (for example, from R&D Systems, Minneapolis, Minn.

Generally methods for generating and producing NT-4/5 polypeptides can also be used for generating and producing BDNF polypeptides.

Identification of trkB Agonists

TrkB agonists (such as antibodies) may be identified using art-recognized methods, including one or more of the following methods. For example, the kinase receptor activation (KIRA) assay described in U.S. Pat. Nos. 5,766,863 and 5,891,650 may be used. This ELISA-type assay is suitable for qualitative or quantitative measurement of kinase activation by measuring the autophosphorylation of the kinase domain of a receptor protein tyrosine kinase (rPTK, e.g. trk receptor), as well as for identification and characterization of potential agonist or antagonists of a selected rPTK. The first stage of the assay involves phosphorylation of the kinase domain of a kinase receptor, in the present case a trkB receptor, wherein the receptor is present in the cell membrane of a eukaryotic cell. The receptor may be an endogenous receptor or nucleic acid encoding the receptor, or a receptor construct, may be transformed into the cell. Typically, a first solid phase (e.g., a well of a first assay plate) is coated with a substantially homogeneous population of such cells (usually a mammalian cell line) so that the cells adhere to the solid phase. Often, the cells are adherent and thereby adhere naturally to the first solid phase. If a “receptor construct” is used, it usually comprises a fusion of a kinase receptor and a flag polypeptide. The flag polypeptide is recognized by the capture agent, often a capture antibody, in the ELISA part of the assay. An analyte, such as a candidate agonist, is then added to the wells having the adherent cells, such that the tyrosine kinase receptor (e.g. trkB receptor) is exposed to (or contacted with) the analyte. This assay enables identification of agonist ligands for the tyrosine kinase receptor of interest (e.g. trkB). Following exposure to the analyte, the adhering calls are solubilized using a lysis buffer (which has a solubilizing detergent therein) and gentle agitation, thereby releasing cell lysate which can be subjected to the ELISA part of the assay directly, without the need for concentration or clarification of the cell lysate.

The cell lysate thus prepared is then ready to be subjected to the ELISA stage of the assay. As a first step in the ELISA stage, a second solid phase (usually a well of an ELISA microtiter plate) is coated with a capture agent (often a capture antibody) that binds specifically to the tyrosine kinase receptor, or, in the case of a receptor construct, to the flag polypeptide. Coating of the second solid phase is carried out so that the capture agent adheres to the second solid phase. The capture agent is generally a monoclonal antibody, but, as is described in the examples herein, polyclonal antibodies or other agents may also be used. The cell lysate obtained is then exposed to, or contacted with, the adhering capture agent so that the receptor or receptor construct adheres to (or is captured in) the second solid phase. A washing step is then carried out, so as to remove unbound cell lysate, leaving the captured receptor or receptor construct. The adhering or captured receptor or receptor construct is then exposed to, or contacted with, an anti-phosphotyrosine antibody which identifies phosphorylated tyrosine residues in the tyrosine kinase receptor. In the preferred embodiment, the anti-phosphotyrosine antibody is conjugated (directly or indirectly) to an enzyme which catalyses a color change of a non-radioactive color reagent. Accordingly, phosphorylation of the receptor can be measured by a subsequent color change of the reagent. The enzyme can be bound to the anti-phosphotyrosine antibody directly, or a conjugating molecule (e.g., biotin) can be conjugated to the anti-phosphotyrosine antibody and the enzyme can be subsequently bound to the anti-phosphotyrosine antibody via the conjugating molecule. Finally, binding of the anti-phosphotyrosine antibody to the captured receptor or receptor construct is measured, e.g., by a color change in the color reagent.

Following initial identification, the agonist activity of a candidate (e.g., an anti-trkB monoclonal antibody) can be further confirmed and refined by bioassays, known to test the targeted biological activities. For example, the ability of a candidate to agonize trkB can be tested in the PC12 neurite outgrowth assay using PC12 cells transfected with full-length trkB (Jian et al., Cell Signal. 8:365-70, 1996). This assay measures the outgrowth of neurite processes by rat pheocytochroma cells (PC12) in response to stimulation by appropriate ligands. These cells express endogenous trka and are therefore responsive to NGF. However, they do not express endogenous trkB and are therefore transfected with trkB expression construct in order to elicit response to trkB agonists. After incubating the transfected cells with the candidate, neurite outgrowth is measured, and e.g., cells with neurites exceeding 2 times the diameter of the cell are counted. Candidates (such as anti-trkB antibodies) that stimulate neurite outgrowth in transfected PC12 cells demonstrate trkB agonist activity.

The activation of trkB may also be determined by using various specific neurons at specific stages of embryonic development. Appropriately selected neurons can be dependent on trkB activation for survival, and so it is possible to determine the activation of trkB by following the survival of these neurons in vitro. Addition of candidates to primary cultures of appropriate neurons will lead to survival of these neurons for a period of at least several days if the candidates activate trkB. This allows the determination of the ability of the candidate (such as an anti-trkB antibody) to activate trkB. In one example of this type of assay, the Nodose ganglion from an E15 mouse embryo is dissected, dissociated and the resultant neurons are plated in a tissue culture dish at low density. The candidate antibodies are then added to the media and the plates incubated for 24-48 hours. After this time, survival of the neurons is assessed by any of a variety of methods. Samples which received an agonist will typically display an increased survival rate over samples which receive a control antibody, and this allows the determination of the presence of an agonist. See, e.g., Buchman et al (1993) Development 118(3):989-1001.

TrkB agonists may be identified by their ability to activate downstream signaling in a variety of cell types that express trkB, either naturally or after transfection of DNA encoding trkB. This trkB may be human or other mammalian (such a rodent or primate) trkB. The downstream signaling cascade may be detected by changes to a variety of biochemical or physiological parameters of the trkB expressing cell, such as the level of protein expression or of protein phosphorylation of proteins or changes to the metabolic or growth state of the cell (including neuronal survival and/or neurite outgrowth, as described herein). Methods of detecting relevant biochemical or physiological parameters are known in the art.

### V. KITS

The invention also provides kits for use in the instant methods. Kits of the invention include one or more containers comprising a purified trkB agonist (for example, a naturally occurring NT-4/5 or BDNF, and an anti-trkB agonist antibody) and instructions for use in accordance with any of the methods of the invention described herein. Generally, these instructions comprise a description of administration of the trkB agonist to treat a disease, such as cachexia, anorexia nervosa, and opioid-induced emesis, according to any of the methods described herein. The kit may further comprise a description of selecting an individual suitable for treatment based on identifying whether that individual has the disease and the stage of the disease.

The instructions relating to the use of trkB agonist generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating a disease described herein (such as cachexia, anorexia nervosa, and opioid-induced emesis). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a trkB agonist. The container may further comprise a second pharmaceutically active agent.

Kits may optionally provide additional components such as buffers and interpretive information. Normally, the kit comprises a container and a label or package insert(s) on or associated with the container.

The following examples are provided to illustrate, but not to limit, the invention.

### EXAMPLES

### Example 1

### Daily NT-4/5 Infusion Resulted in Body Weight Gain and Hyperphagia in Obese Baboons

Three obese female baboons (body weight ranges 20-30 kg) received intravenous (IV) infusion of human NT-4/5 at 2 mg/kg once per day from day 1 to day 24. Three additional obese female baboons (body weight ranges 20-30 kg) received IV vehicle (PBS) infusion once per day from day 1 to day 24. Food intake was measured daily for the first 45 days. The animals were weighed once a week for the first 53 days and then followed up on day 81 and day 109 respectively.

FIG. 1 shows the effect of daily NT-4/5 infusion on body weight in obese baboons. As shown in FIG. 1, body weight of the NT-4/5 treated group was significantly increased as compared to the vehicle group; and body weight in the NT-4/5 treated group returned to the level of the vehicle group by day 81. The data indicated that daily NT-4/5 infusion resulted in prolonged but reversible body weight gain in obese baboons.

FIG. 2 shows the effect of daily NT-4/5 infusion on food intake in obese baboons. As shown in FIG. 2, food intake in the NT-4/5 treated group was significantly increased as compared to the vehicle group; and food intake in the NT-4/5 treated group returned to the level of the vehicle group by day 33. The data indicated that daily NT-4/5 infusion resulted in reversible hyperphagia in obese baboons.

### Example 2

### Twice Per Week NT-4/5 Infusion Resulted in Body Weight Gain but No Hyperphagia in Obese Baboons

Three obese female baboons (body weight ranges 20-30 kg) received intravenous (IV) infusion of human NT-4/5 at 2 mg/kg twice per week from day 1 to day 39. Three additional obese female baboons (body weight ranges 20-30 kg) received IV vehicle (PBS) infusion twice per week from day 1 to day 39. Food intake was measured daily for the first 55 days. The animals were weighed weekly for the first 66 days and then followed up on day 94 and day 122 respectively.

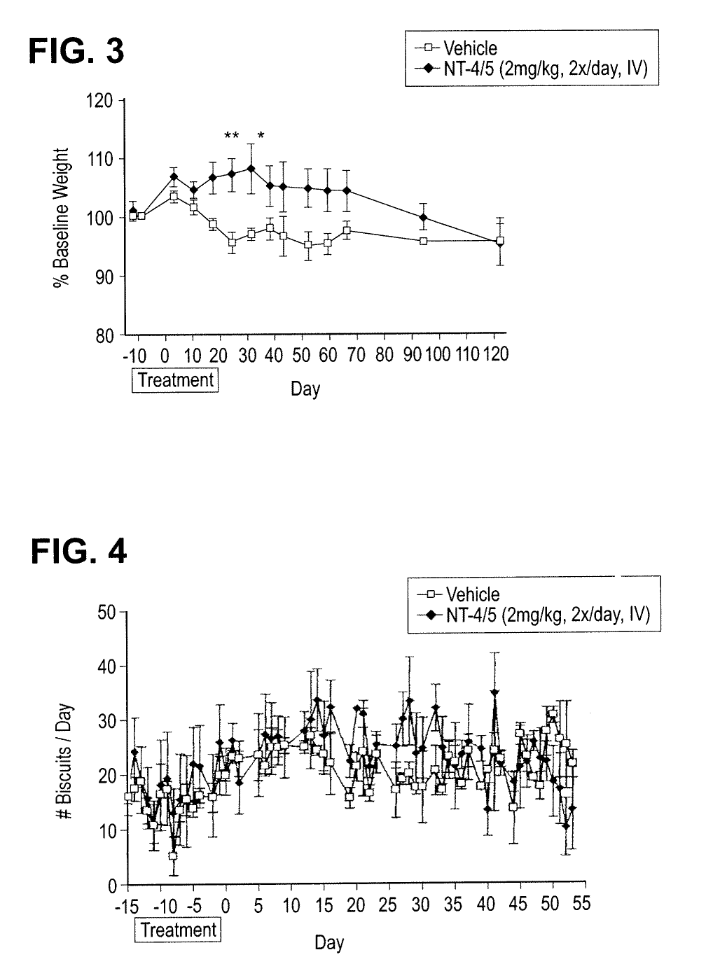

FIG. 3 shows the effect of twice per week NT-4/5 infusion on body weight in obese baboons. As shown in FIG. 3, body weight of the NT-4/5 treated group was significantly increased as compared to the vehicle group; and body weight in the NT-4/5 treated group returned to the level of the vehicle group by day 94. The data indicated that twice per week NT-4/5 infusion resulted in reversible body weight gain in obese baboons.

FIG. 4 shows the effect of twice per week NT-4/5 infusion on food intake in obese baboons. As shown in FIG. 4, twice per week NT-4/5 infusion did not significantly change food intake in obese baboons by two way ANOVA analysis. Bonferroni posttests analysis of data did not show significant pairwise difference between the NT-4/5 group (solid triangles) with the vehicle control group (open squares).

### Example 3

### Daily NT-4/5 Infusion Resulted in Body Weight Gain and Hyperphagia in Lean Cynomolgus Monkeys

Three lean female cynomolgus monkeys (body weight ranges 3-5 kg) received intravenous (IV) infusion of human NT-4/5 at 2 mg/kg daily from day 1 to day 31. Three lean female cynomolgus monkeys (body weight ranges 3-5 kg) received IV pegylated NT-4/5 (pegylated NT4-G1S) at 0.6 mg/kg infusion once per week from day 1 to day 31. Pegylated NT-4/5 was generated by introducing a mutation of the glycine 1 position of human NT-4/5 mature sequence to serine and attaching a PEG to the first amino acid serine as described in Example 7 of U.S. Pub. No. 2005/0209148 and PCT WO 2005/082401. Three additional lean female cynomolgus monkeys (body weight ranges 3-5 kg) received IV vehicle (PBS) infusion once per day from day 1 to day 31. Food intake was measured daily for the first 50 days. The animals were weighed once a week up to day 50.

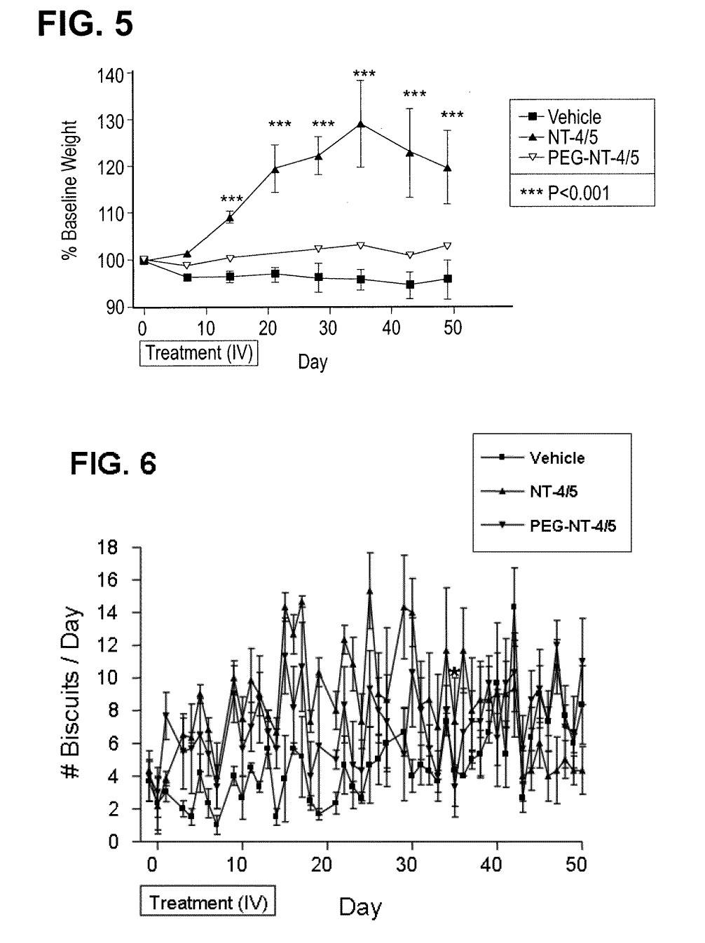

FIG. 5 shows the effect of daily NT-4/5 infusion on body weight in lean female cynomolgus monkeys. As shown in FIG. 5, body weight of the daily NT-4/5 treated group, but not the weekly pegylated NT-4/5, was significantly increased as compared to the vehicle group. The body weight of the NT-4/5 treated group had not yet fully returned to the level of the vehicle group. The data indicated that daily NT-4/5 infusion resulted in body weight gain in lean cynomolgus monkeys.

FIG. 6 shows the effect of daily NT-4/5 infusion on food intake in lean cynomolgus monkeys. As shown in FIG. 6, food intake in the daily NT-4/5 treated group, but not the weekly pegylated NT-4/5 treated group was significantly increased as compared to the vehicle group; and food intake in the NT-4/5 treated group returned to the level of the vehicle group by day 38. The data indicated that daily NT-4/5 infusion resulted in reversible hyperphagia in lean cynomolgus monkeys.

The effects of NT-4/5 and pegylated NT-4/5 on body weight were also tested by subcutaneous administration. Three lean female cynomolgus monkeys (body weight ranges 3-5 kg) received subcutaneous (SC) injection of human NT-4/5 at 2 mg/kg daily from day 1 to day 21. Three lean female cynomolgus monkeys (body weight ranges 3-5 kg) received SC injection of pegylated NT-4/5 at 1 mg/kg injection once per day from day 1 to day 21. Three additional lean female cynomolgus monkeys (body weight ranges 3-5 kg) received SC injection of vehicle (PBS) once per day from day 1 to day 21. The animals were weighed once a week up to day 21.

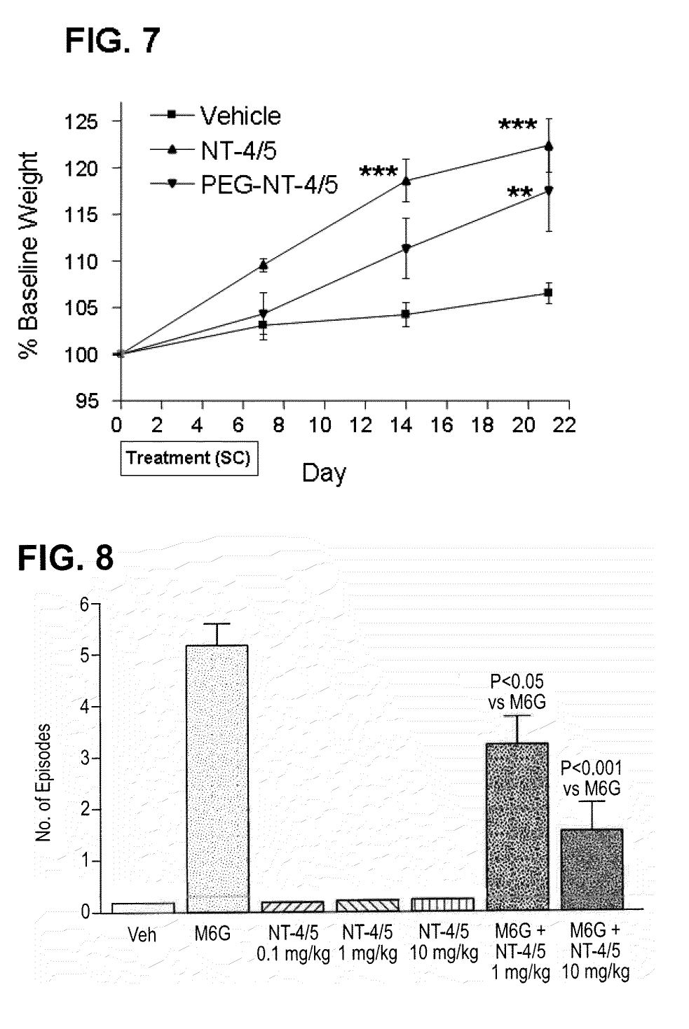

FIG. 7 shows the effect of daily SC injection of NT-4/5 and pegylated NT-4/5 on body weight in lean female cynomolgus monkeys. As shown in FIG. 7, body weight of the NT-4/5 treated group was significantly increased as compared to the vehicle group. In addition, body weight of the pegylated NT-4/5 treated group was also significantly increased as compared to the vehicle group.

### Example 4

### Daily Subcutaneous Injection of NT-4/5 Showed No Significant Effect on Body Weight and Food Intake in NZW Rabbits

Five male and five female New Zealand White (NZW) rabbits (body weight ranges 3-4 kg) received subcutaneous (SC) injection of human NT-4/5 at 2 mg/kg daily from day 1 to day 15. Five additional male and five female NZW rabbits (body weight ranges 3-5 kg) received SC injection of vehicle (PBS) once per day from day 1 to day 15. Food intake was measured daily for the first 15 days. The animals were weighed once a week up to day 15.

No statistically significant differences were observed between the NT-4/5 treated group and the vehicle group for body weight or food intake. The comparisons were made between the NT-4/5 treated male rabbits and the vehicle male rabbits, and between the NT-4/5 treated female rabbits and the vehicle female rabbits.

### Example 5

### Single Injection of NT-4/5 Did not Cause Vomiting but can Reduce Morphine-Induced Vomiting in Ferrets

Effect of NT-4/5 on emesis was studies in the adult female ferrets with body weight of about 1 kg (Marshall Farm, Conn.). An emetic agent (0.05 mg/kg of morphine 6-glucuronide, M6G) was given subcutaneously as a positive control to establish the baseline prior to NT-4/5 administration. An increasing dose of NT-4/5 (0.1, 1, or 10 mg/kg) was injected subcutaneously to 6 ferrets (for each dosage) alone to test whether NT-4/5 could cause any adverse effects such as retching or vomiting. In addition, two doses, 1 mg/kg and 10 mg/kg, of NT-4/5 were given 10 minutes before M6G to test if NT-4/5 could suppress M6G induced emesis. The animals were returned to the home cage and observed for the latency, the number of retches and of vomits over a period of 60 min post injection.

As shown in FIG. 8, single injection of 0.1, 1 or 10 mg/kg NT-4/5 alone did not cause vomiting in the ferrets, while a single SC injection of 0.05 mg/kg M6G effectively induced emesis. Both 1 mg/kg and 10 mg/kg of NT-4/5 significantly reduced M6G-induced vomiting in ferrets.

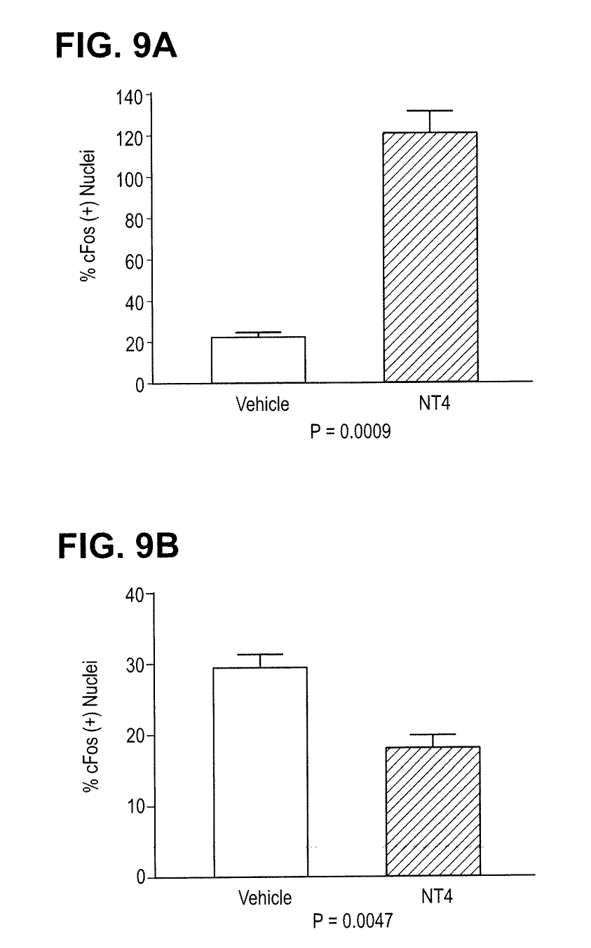

To test the trkB activation site that might be responsible for anti-emesis effect of injection of NT-4/5 in ferret, c-Fos activation of trkB in the ferret brainstem was tested. A single dose of 10 mg/kg of NT-4/5 was injected subcutaneously, followed by intravenous 10 mg/kg cisplatin 5 minutes later, to five female ferrets. A single dose of vehicle injection, followed by cisplatin 5 minutes later, was given to an additional four female ferrets as a negative control. The animals were sacrificed 1 hour later by pentobarbital sodium (65 mg/kg ip), fixed by intracardial perfusion with 1 L/kg of PBS followed by 1 L/kg 4% paraformaldehyde, pH7.3. Sections of brain stem were cut at 30 um and floating sections were incubated in 10% normal donkey serum (NDS) diluted in 0.1% Triton X-100 (in PBS) for 1 h followed by incubation in sheep anti-Fos (1:1,000, OA-11-824, Genosys Biotechnologies, Cambridge, UK) in PBS with 0.1% Triton X-100 and 10% NDS for 48 h at 4° C. Sections were washed in PBS and then incubated in biotinylated anti-sheep IgG secondary antibody solution for 60 min at room temperature. Staining was revealed by using the avidin biotin complex technique (Vectastain Elite avidinbiotin complex (ABC) Kit, Vector). Briefly, sections were incubated in ABC reagent for 60 min at room temperature and then in a solution containing 3,3-diaminobenzidine (0.5 mg/ml) for 30-60 s. Brain stem sections were then mounted on slides to dry for 24 h, dehydrated for 4 min each in 50, 70, 95, and 100% ethanol, and then cleared in xylene, after which they were mounted and viewed. The boundaries of the nuclei and subnuclei of the nucleus tractus solitarius (NTS) were assessed in adjacent sections stained with cresyl violet. The number of c-Fos immunoreactive neuronal nuclei was determined bilaterally for the area postrema, doral vagal nucleus (DMNX), and all subnuclei of the NTS at three levels along the rostrocaudal extent of the dorsal vagal complex (DVC), 0.5-1.0 mm rostral and 0.5 mm caudal to obex, and at obex. Three sections per level per animal were counted and averaged. Data were compared by using ANOVA with Tukey's post test (Prism; GraphPadSoftware, San Diego, Calif.). NT-4/5 treatment dramatically increased the number of c-Fos positive nuclei in the area postrema compared to the vehicle injected animals (FIG. 9A, P=0.0009, Student's t-test). In contrast, NT-4/5 treatment significantly decreased the number of c-Fos positive nuclei in the dorsal vagal nucleus compared to the vehicle injected animals (FIG. 9B, P=0.0047, Student's t-test). On the other hand, NT-4/5 treatment did not significantly alter the number of c-Fos positive nuclei in other brainstem nuclei, including the multiple subclei of the NTS as well as the paraventricular nuclei of the hypothalamus.

Area postrema, unlike most of the other brain areas, lie outside of the blood brain barrier and have full access to the circulating macromolecules (for a recent example, see Yang and Ferguson, 2003, Regul. Pept. 112(1-3):9-17). The induction of c-Fos is a known immediate early event of trkB activation by its ligands such as BDNF and NT-4/5 (Ip et al. 1993, J. Neurosci. 13(8):3394-405 and Marsh et al. 1993, J. Neurosci. 13(10): 4281-92).

These data together suggest that area postrema constitute at least in part a bona fide “peripherally accessible” target of systemically delivered NT-4/5 or other trkB agonists. The reduction of c-Fos expression in the dorsal vagal nucleus (FIG. 9B) may reflect partial attenuation of the vomiting circuit by NT-4/5 pre-treatment.

### Example 6

### Generation and Screening of trkB Agonist Antibodies

Immunization for generating monoclonal anti-TrkB agonist antibodies: A single Balb/C mouse was injected 5 times on a regular schedule with 8 ug of human TrkB extracellular domain as antigen. His-tagged human TrkB extracellular domain (residues 31-430) was expressed using vector pTriEx-2 Hygro (Novagen, Madison Wis.) in 293 cells. TrkB extracellular domain was purified using Ni-NTA resin via manufacturer's instructions (Qiagen, Valencia, Calif.). For the first 4 injections, antigen was prepared by mixing human TrkB with RIBI adjuvant system and alum. Eight ug total of antigen was given via injection to the scruff of the neck, the foot pads and IP, approximately every 3 days over the course of 11 days. On Day 13, the mouse was euthanized and the spleen was removed. Lymphocytes were fused with 8653 cells to make hybridoma clones. Clones were allowed to grow then selected as anti-TrkB positives by ELISA screening with both Human and Rat TrkB ELISA.

ELISA screening anti-trkB antibodies: Supernatants from growing hybridoma clones were screened for their ability to bind both human and rat TrkB. The assays were performed with 96-well plates coated overnight with 100 ul of 0.5 ug/ml rat or human TrkB-Fc fusion protein. Excess reagents were washed from the wells between each step with PBS containing 0.05% Tween-20. Plates were then blocked with phosphate buffered saline (PBS) containing 0.5% BSA. Supernatant was added to the plates and incubated at room temperature for 2 hours. Horse radish peroxidase (HRP) conjugated goat-anti mouse Fc was added to bind to the mouse antibodies bound to TrkB. Tetramethyl benzidine was then added as substrate for HRP to detect amount of mouse antibody present in the supernatant. The reaction was stopped and the relative amount of antibody was quantified by reading the absorbance at 450 nm. Fifty antibodies were shown positive in the ELISA assay. Among these antibodies, five were further tested and shown to have agonist activity. See Table 2 below.

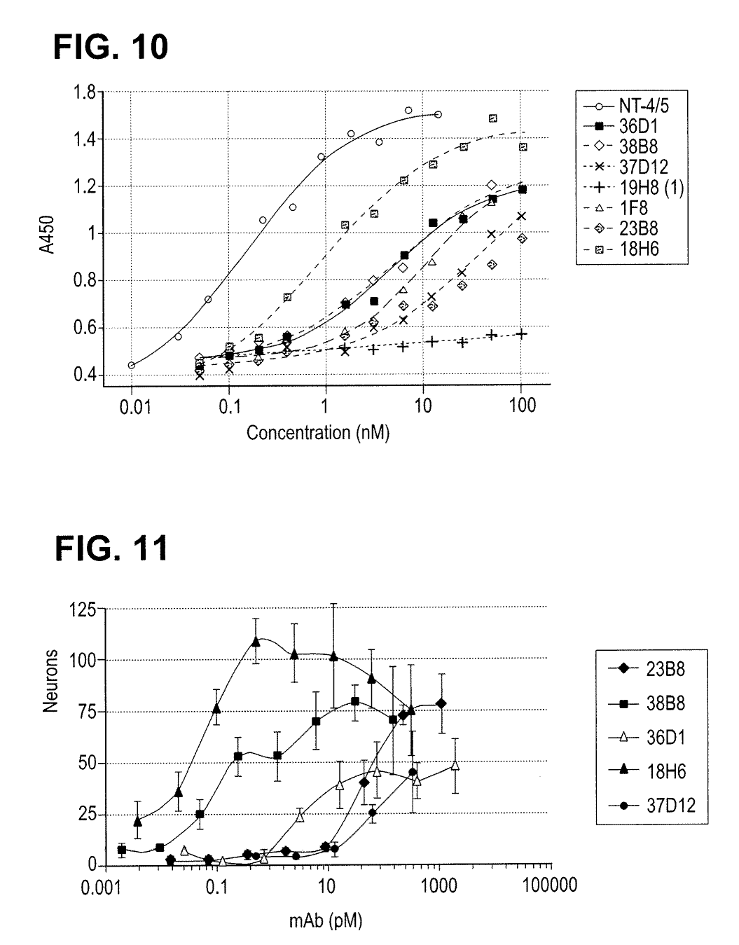

KIRA Assay: This assay was used to screen antibodies found positive in the ELISA for the ability to induce receptor tyrosine kinase activation for human TrkB. Sadick et. al. (1997) Experimental Cell Research 234(2):354-61. Utilizing a stable cell line transfected with gD tagged human TrkB, purified murine antibodies from the hybridoma clones were tested for their ability to activate the receptor on the surface of the cells similar to the activation seen with the natural ligands, BDNF and NT-4/5. Natural ligand induced self phosphorylation of the kinase domain of the TrkB receptor. After the cells were exposed to various concentrations of the antibodies, they were lysed and an ELISA was performed to detect phosphorylation of the TrkB receptor. EC50 (shown in Table 2 below and FIG. 10) was determined for each putative TrkB agonist and was compared to that of the natural ligand NT-4/5.

E15 Nodose neuron survival assay: The Nodose ganglion neurons obtained from E15 embryos were supported by BDNF, so that at saturating concentrations of the neurotrophic factor the survival was close to 100% by 48 hours in culture. In the absence of BDNF, less than 5% of the neurons survived by 48 hours. Therefore, the survival of E15 nodose neurons is a sensitive assay to evaluate the agonist activity of anti-TrkB antibodies, i.e. agonist antibodies will promote survival of E15 nodose neurons.

Time-mated pregnant Swiss Webster female mice were euthanized by CO₂ inhalation. The uterine horns were removed and the embryos at embryonic stage E15 were extracted. The nodose ganglia were dissected then trypsinized, mechanically dissociated and plated at a density of 200-300 cells per well in defined, serum-free medium in 96-well plates coated with poly-L-ornithine and laminin. The agonist activity of anti-TrkB antibodies was evaluated in a dose-response manner in triplicates with reference to human BDNF. After 48 hours in culture the cells were subjected to an automated immunocytochemistry protocol performed on a Biomek FX liquid handling workstation (Beckman Coulter). The protocol included fixation (4% formaldehyde, 5% sucrose, PBS), permeabilization (0.3% Triton X-100 in PBS), blocking of unspecific binding sites (5% normal goat serum, 0.1% BSA, PBS) and sequential incubation with a primary and secondary antibodies to detect neurons. A rabbit polyclonal antibody against the protein gene product 9.5 (PGP9.5, Chemicon), which was an established neuronal phenotypic marker, was used as primary antibody. Alexa Fluor 488 goat anti-rabbit (Molecular Probes) was used as secondary reagent together with the nuclear dye Hoechst 33342 (Molecular Probes) to label the nuclei of all the cells present in the culture. Image acquisition and image analysis were performed on a Discovery-1/GenII Imager (Universal Imaging Corporation). Images were automatically acquired at two wavelengths for Alexa Fluor 488 and Hoechst 33342, with the nuclear staining being used as reference point, since it is present in all the wells, for the image-based auto focus-system of the Imager. Appropriate objectives and number of sites imaged per well were selected to cover the entire surface of each well. Automated image analysis was set up to count the number of neurons present in each well after 48 hours in culture based on their specific staining with the anti-PGP9.5 antibody. Careful thresholding of the image and application of morphology and fluorescence intensity based selectivity filters resulted in an accurate count of neurons per well. EC50s (shown in Table 2 below and FIG. 11) were determined for each putative TrkB agonist antibody and were compared to that of the natural ligands.

Table 2 below shows the five anti-trkB antibodies identified and their activities on mouse neuron survival and phosphorylation activity on human trkB.

TABLE 2

| | | | Mouse Neuron | Human KIRA |
|---|---|---|---|---|
| | HuTrkB | RatTrkB | survival Assay | Assay |
| Clone | ELISA | ELISA | (estimated EC₅₀) | (EC₅₀) |
| 18H6 | + | + | 0.01 pM | 0.5 nM |
| 38B8 | + | + | 0.2 pM | 5 nM |
| 36D1 | + | + | 5 pM | 5 nM |
| 37D12 | + | + | 50 pM | 56 nM |
| 23B8 | + | + | 11 pM | 50 nM |

Intracranial Injections of anti-trkB agonist antibodies to mice: Male C57B6 retired breeder mice (aged 8-12 months) were obtained from Charles River Laboratories (Hollister facility) and allowed to acclimate in a temperature/humidity-controlled environment, with a 12 hour light/dark cycle, with ad libitum access to food and water for at least 5 days before injection. Each mouse was anaesthetized with isoflurane, to clip a section of hair above the skull. The mouse was fixed onto the stereotaxic surgery instrument (Kopf model 900), anaesthetized, and kept warm with an electric heating pad set to medium. Betadine was rubbed onto the shaved portion of the skull to sterilize the region. A small median-longitudinal incision of about 1 cm long was made above the cranium starting just behind the ears towards the eyes. The skull was revealed, and a circular space of about 1 cm in diameter of the skull surface was cleaned with a cotton swab to remove any connective tissue. The surface was cleaned with a cotton swab dipped in 30% hydrogen peroxide, to reveal the Bregma. Using the drill tip as a probe to measure skull depth, the cranium was adjusted horizontally and vertically to insure that it was level before drilling. Deviation of depth (zeroed at the Bregma), from 0.5 mm medial compared to 0.5 mm lateral, as well as 0.5 mm anterior compared to 0.5 mm posterior, was minimized to within a difference of ±0.05 mm. According to the mouse brain atlas (Franklin, K. B. J. & Paxinos, G., The Mouse Brain in Stereotaxic Coordinates. Academic Press, San Diego, 1997), coordinates for a single, lateral, intrahypothalamic injection were as follows: 1.30 mm posterior from the Bregma; −0.5 mm from midline; Depth, 5.70 mm from the surface of the skull (at the Bregma). A small hole was drilled through the skull, avoiding contact with the brain. The drill was replaced with a beveled 26 gauge needle attached to a Hamilton syringe (model 84851) and returned to the same coordinates. 2 ul of compound was injected into the lateral hypothalamus incrementally over the course of 2 minutes. The needle was kept at this position for 30 seconds after injection, then raised 1 mm. After another 30 seconds, the needle was raised 1 mm. 30 seconds later, the needle was completely removed. The incision was then closed and held together with 2-9 mm wound clips (Autoclip, Braintree Scientific, Inc.). The injection was performed on day 0. Body weight and food intake were monitored daily until day 15.

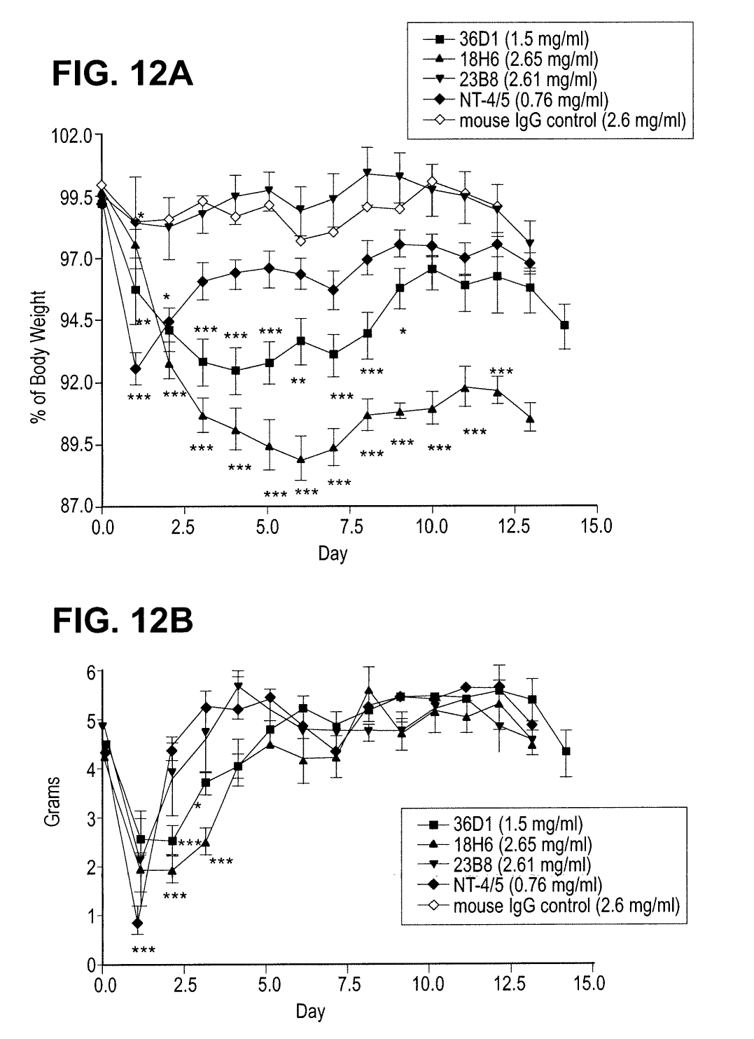

As shown in FIG. 12A and FIG. 12B, intracranial injections of antibody 18H6 and 36D1 at the specified dose significantly reduced body weight and food intake in mice. The control IgG antibody and 23B8, given at the specified dose, did not significantly affect either the food intake or the body weight. Two way ANOVA with Bonferroni posttests was used for statistical analysis. This indicates that anti-trkB agonist antibodies have an effect on body weight and food intake qualitatively similar to NT-4/5, a natural trkB agonist, when injected directly to the CNS.

### Example 7

### Peripheral Injection of trkB Agonist Antibody Resulted in Increased Food Intake and Body Weight in Monkeys

Adult lean, female cynomolgus monkeys (weighing 3-5 kg at baseline) received intravenous injections of mouse monoclonal agonist antibody 38B8 and the other three animals received vehicle twice a week. Food consumption was monitored daily and body weight was monitored weekly. The statistical analyses were performed by using PRISM (GraphPad Software Inc., San Diego, Calif.). All data and graphs were expressed in mean±standard error of mean (SEM). The data were analyzed by 2-way ANOVA with Dunnet's post tests (* P<0.05, ** P<0.01, *** P<0.001).

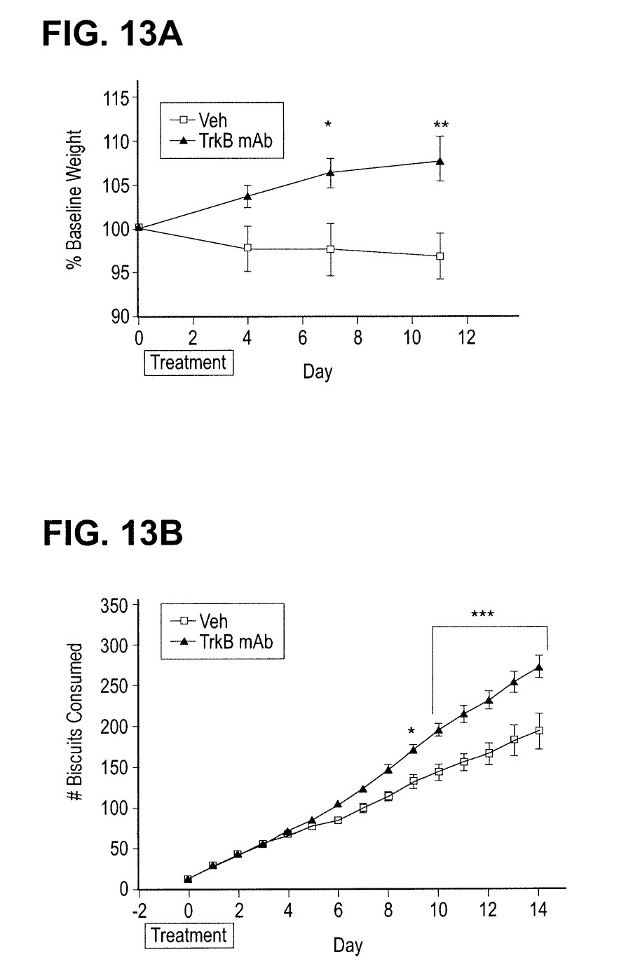

The monkeys that were treated twice a week injections of 5 mg/kg of the trkB agonist antibody 38B8 exhibited 40% increase in cumulative food intake (FIG. 13A) and 10% increase in weight (FIG. 13B) within 2 weeks, indicating that specific activation of the trkB tyrosine kinase receptor mediates orexigenic effects and reduces body weight.

It is understood that the examples and embodiments described herein are for illustrative purposes only and that various modifications or changes in light thereof will be suggested to persons skilled in the art and are to be included within the spirit and purview of this application. All publications, patents and patent applications cited herein are hereby incorporated by reference in their entirety for all purposes to the same extent as if each individual publication, patent or patent application were specifically and individually indicated to be so incorporated by reference.

What is claimed is:

1. A method for treating cachexia in a primate comprising peripherally administering an effective amount of NT-4/5 to a primate suffering from or in need of preventing cachexia, thereby ameliorating or preventing one or more symptoms of cachexia.

2. The method of claim 1, wherein said primate is a human.

3. The method of claim 1, wherein the cachexia is associated with cancer.

4. The method of claim 1, wherein the cachexia is associated with AIDS.

5. A method for treating unwanted weight loss in a primate, comprising peripherally administering an effective amount of NT-4/5 to a primate suffering from or in need of preventing unwanted weight loss, thereby ameliorating or preventing one or more symptoms of unwanted weight loss.

6. The method of claim 5, wherein said primate is a human.

7. The method of claim 5, wherein the unwanted weight loss is associated with cancer.

8. The method of claim 5, wherein the unwanted weight loss is associated with AIDS.

9. A method for treating opioid-induced emesis in a mammal comprising peripherally administering an effective amount of NT-4/5 to a primate suffering from opioid-induced emesis, thereby ameliorating one or more symptoms of opioid-induced emesis.

10. The method of claim 9, wherein said mammal is a human.

11. A method for treating anorexia nervosa in a primate comprising peripherally administering an effective amount of NT-4/5 to a primate suffering from anorexia nervosa, thereby ameliorating one or more symptoms of anorexia nervosa.

12. The method of claim 11, wherein said primate is a human.
